(19) United States
(12) Patent Application Publication
(10) Pub. No.: US 2015/0310573 A1
(43) Pub. Date: Oct. 29, 2015

(54) PROVIDING HEALTHCARE SOLUTIONS AND WORKFLOW MANAGEMENT

(71) Applicant: James Grant, Sewickiey, PA (US)

(72) Inventor: James Grant, Sewickiey, PA (US)

(73) Assignee: PROVIDING HEALTHCARE SOLUTIONS AND WORKFLOW MANAGEMENT ELIZUR CORPORATION

(21) Appl. No.: 14/007,945

(22) PCT Filed: Mar. 30, 2012
(86) PCT No.: PCT/US12/31362

§ 371 Date: Dec. 9, 2013

Related U.S. Application Data
(60) Provisional application No. 61/470,843, filed on Apr. 1, 2011.

(51) Int. Cl. G06Q 50/22; G06Q 10/08; G06Q 10/06

(52) U.S. Cl. CPC G06Q 50/22; G06Q 10/06315; G06Q 10/087

(57) ABSTRACT

One aspect provides a method, including: operating a computing device to, in response to an input physician identifier and an input diagnosis identifier, select a musculoskeletal treatment protocol, the musculoskeletal treatment protocol including at least one of the following: a predetermined musculoskeletal medical product and a predetermined musculoskeletal treatment service; operating the computing device to, responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and operating the computing device to fill at least a portion of the order corresponding to the musculoskeletal treatment protocol. Other embodiments are disclosed.

### CLAIM FOR PRIORITY

This application claims priority to U.S. Provisional Application No. 61/470,843, filed Apr. 1, 2011, which is incorporated by reference herein.

### BACKGROUND

Conventionally, when a patient seeks to receive examination and treatment for injuries or ailments, it is necessary for the patient to make an appointment with a physician and take a trip to the physician's office. Alternatively when an injury is severe, the patient must take a trip to a hospital, an urgent care facility, or an emergency room. In addition to having to make a trip the physician's office or hospital, the patient must either wait for the appointment date to arrive or alternatively must endure long wait times at the emergency room. After a diagnosis is finally obtained, a variety of procedures must be followed in order to actually formulate and deliver a treatment plan. A lack of coordination results in poor resource utilization. For example, traditional methods of handling prescriptions for medical devices and other treatments or therapies are not the most time efficient nor do they offer the greatest amount of flexibility or integration.

### BRIEF SUMMARY

In summary, one aspect provides a computer program product, comprising: a computer readable storage medium having computer readable program code embodied therewith, the computer readable program code comprising: computer readable program code configured to, in response to an input physician identifier and an input diagnosis identifier, select a musculoskeletal treatment protocol, the musculoskeletal protocol including at least one of the following: a predetermined musculoskeletal medical product and a predetermined musculoskeletal treatment service; computer readable program code configured to, responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and computer readable program code configured to fill at least a portion of the order corresponding to the musculoskeletal treatment protocol.

Another aspect provides a method, comprising: operating a computing device to, in response to an input physician identifier and an input diagnosis identifier, select a musculoskeletal treatment protocol, the musculoskeletal treatment protocol including at least one of the following: a predetermined musculoskeletal medical product and a predetermined musculoskeletal treatment service; operating the computing device to, responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and operating the computing device to fill at least a portion of the order corresponding to the musculoskeletal treatment protocol.

A further aspect provides an apparatus, comprising: one or more processors; a storage medium in connection with the one or more processors, the storage medium storing a program of instructions that when executed by the one or more processors causes the apparatus to: in response to an input physician identifier and an input diagnosis identifier, select a musculoskeletal treatment protocol, the musculoskeletal treatment protocol including at least one of the following: a predetermined musculoskeletal medical product and a predetermined musculoskeletal treatment service; responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and fill at least a portion of the order corresponding to the musculoskeletal treatment protocol.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example system architecture;

FIG. 2(A-G) illustrates an example method of selecting/customizing a treatment plan based on a diagnosis;

FIG. 3 is a flow diagram illustrating an example process for provisioning a treatment plan based on a virtual visit;

FIG. 4 is a flow diagram illustrating an example process for provisioning a treatment plan based on a virtual visit;

FIG. 5(A-G) illustrates examples of patient agreement formation and order conversion processing;

FIG. 6 is a flow diagram illustrating an example of predicting expected cash flow;

FIG. 7 is a flow diagram illustrating an example of automating billing selections;

FIG. 8(A-D) illustrates examples of claim processing;

FIG. 9(A-C) illustrates examples of inventory processing;

FIG. 10(A-C) illustrates examples of rental product management;

FIG. 11 is a flow diagram illustrating an example customer satisfaction survey process;

FIG. 12 is a flow diagram illustrating an example public interface for entering a diagnosis and receiving a treatment plan;

FIG. 13(A-G) illustrates examples of automated healthcare and workflow processing;

FIG. 14(A-C) illustrate examples of a public/patient interface for obtaining a diagnosis and modification of the interface.

FIG. 15 illustrates an example computing system.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail to avoid obfuscation. The following description is intended only by way of example, and simply illustrates certain example embodiments.

Embodiments provide an electronic platform (for example implemented on an electronic device such as a server, a workstation or the like) with which a user (such as a healthcare provider) may manage patient care, for example during a consultation following an examination by a physician or healthcare provider. The electronic platform takes a diagnosis as input and facilitates provisioning of a treatment plan, through and including selecting a protocol for treatment, including products and/or services, producing a patient agreement and an order, handling prescriptions, managing billing (both patient billing and insurer/payor billing), evaluating effectiveness of treatment plans, including customer surveys and revenue management, and providing follow up care. An embodiment facilitates alternative means for obtaining a diagnosis, such as providing a public interface facilitating a virtual visit for obtaining a diagnosis. Some example embodiments provide a platform that has been customized for orthopedic/musculoskeletal related patient care management.

A platform consistent with embodiments assists in managing patient care by allowing a user (such as a healthcare provider) to provide one or more treatment plans for a patient based on a variety of factors, such as a particular diagnosis provided to the patient by a physician or other healthcare provider. In this respect, an embodiment provides for selecting an appropriate treatment plan, including a protocol, given a specific diagnosis.

A protocol may form part of a treatment plan. A user, for example a physician or other healthcare provider, may select a protocol based on a patient examination that leads to a diagnosis. The user may access the platform that stores protocols, such as default/standardized protocols and/or protocols customized by a particular physician or healthcare provider for a particular patient or patient type. A protocol is a specific regimen signed off on by a physician or other healthcare provider for treating a given diagnosed condition, which may include healthcare treatment services and/or products. Treatment services, for example, may include physical therapy, pharmacy services, and case management services. Products, for example, may include musculoskeletal products which are either internal or external to the body. Examples of internal products are replacement joints, sutures, and other products utilized by a surgeon during surgery. Examples of external products include bracing, supports (e.g. crutches and canes), motion devices for rehabilitation, and pharmacological agents. A choice in protocol may also be influenced by previous medical history. For example, if a patient has a rotator cuff injury, the fact that the patient had previously broken the humorous of the affected arm would influence the protocol for the rotator cuff injury. For example, an orthopedist may examine a patient (in person or virtually), make a diagnosis, and select a protocol for the patient based on the diagnosis. The physician may select a standard/default protocol, having reviewed the standard/default protocol and found it satisfactory, or alternatively the physician may customize a protocol to suit a particular need. Thus, a user of the platform may select an appropriate protocol for a given patient having a given diagnosis.

One or more treatment services may form part of a treatment plan. The one or more services may be called for by a protocol. A user, such as a healthcare consultant, may select one or more treatment services, such as physical therapy in the case of a carpal tunnel diagnosis, as indicated in a carpal tunnel protocol. For example, for a given patient with a diagnosis of carpal tunnel, a particular protocol selected by the patient's physician or healthcare provider may indicate that physical therapy is medically necessary. Several physical therapy treatment services alternatives may qualify as appropriate under a given protocol. The platform may in such a circumstance provide information such as an automatically gathered list of suitable physical therapy treatment services for the given diagnosis from which a user may choose. In this regard, the platform may facilitate selection of a physical therapy treatment service consistent with the treatment plan, a protocol or the like. As part of facilitating selection of treatment services, an embodiment may maintain and provide treatment services vendor information, such as relevant scheduling information, how much the treatment services cost, if the treatment services are covered by a particular insurance provider or plan relevant to the patient, et cetera.

One or more products may form part of a treatment plan. The one or more products may be called for by a protocol. A user, such as a healthcare consultant, may select one or more products, such as a particular prescription medication, or durable medical product/equipment such as a brace in the case of a carpal tunnel diagnosis. For example, for a given patient with a diagnosis of carpal tunnel, a particular protocol selected by the patient's physician or healthcare provider may indicate that a brace is medically necessary. Several braces may qualify as appropriate under a given protocol. The platform may in such a circumstance provide information such as an automatically gathered list of suitable braces for the given diagnosis from which a user may choose. In this regard, the platform may facilitate selection of products consistent with the treatment plan, a protocol or the like. As part of facilitating selection of products, an embodiment may maintain and provide inventory information, such as if a particular product is currently in stock, if the product must be ordered, how much the product costs, if the product is covered by a particular insurance provider or plan relevant to the patient, et cetera. In a circumstance in which the product is a prescription product (pharmaceutical, medical device/equipment, et cetera), an embodiment may facilitate electronic handling of a prescription, as further described herein.

A platform consistent with embodiments may automate patient information gathering, patient billing, and related services. For example, for a particular patient having a particular diagnosis and protocol, a treatment plan may be managed at least in part in terms of patent specific billing or insurance coverage information. In a case where a patient has insurance coverage, the patient may indicate an insurance identification number. Using the insurance identification number, or like identification, the platform may automatically populate data fields relevant to billing, such as name, gender, age, et cetera, as gathered from a relevant data source (for example, via contacting a data server of an insurer). Moreover, the platform, given the selections made for products and/or services, may make predictions as to whether specific portions of the treatment plan, such as products or services, are likely to be covered by a particular insurer, what the projected costs will be, and even suggest alternatives (such as suggesting an alternative product or service, or an alternative billing code therefore, if a previously selected product or service is likely not covered).

A platform consistent with embodiments may automate formalization of patient agreements. For example, responsive to a diagnosis, selection of a treatment plan, including a protocol, product(s) and/or service(s), a patient may be presented with agreement information in electronic form, which may also include educational information. An embodiment provides a means to facilitate electronic patient agreement formation by presenting such information to the patient electronically, such as on a tablet computing device, and allowing for capturing of a patient's signature electronically.

An embodiment provides an electronic platform (for example implemented on an electronic device such as a tablet computer) that facilitates management of healthcare via providing applications to oversee inventory and rentals regarding durable medical products/equipment, managing billing and revenue, and analyzing user satisfaction, as further described herein.

Another embodiment provides a healthcare management tool with a public interface, such as via providing a publicly available interactive web site or downloadable application (app), in which a user (patient) may receive physician or healthcare provider examination and/or consultation services in near real time. The public healthcare tool may provide a user (patient) with an anatomical navigation interface in which a detailed series of anatomical animations, used in combination with hierarchical questions, allow the user (patient) to pinpoint a specific anatomical area and issue on which the examination/consultation should focus. In response to the user (patient) providing sufficient information to select an appropriate physician or healthcare provider, such as via an anatomical navigation interface, an embodiment may connect the user (patient) and physician or healthcare provider, such as via live chat and/or video conferencing, such that a virtual examination/consultation may take place. The virtual examination/consultation may be prefaced by requiring the user (patient) to have a pre-existing relationship with the physician, healthcare provider, or healthcare organization providing the virtual examination/consultation, as ascertained via a login or similar mechanism. The virtual examination/consultation may result in a diagnosis, which may be handled by various embodiments in a variety of ways, as further described herein.

The illustrated example embodiments will be best understood by reference to the figures. The following description is intended only by way of example, and simply illustrates certain example embodiments.

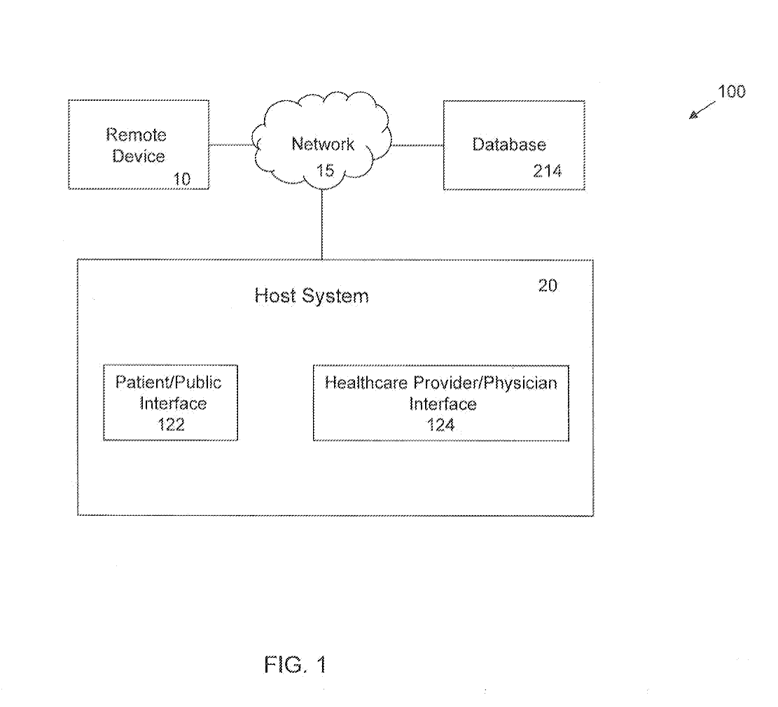

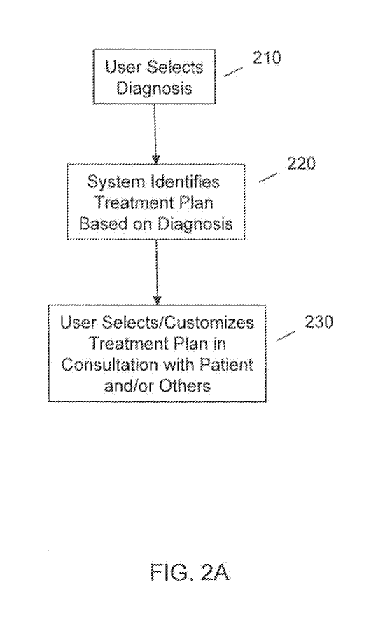

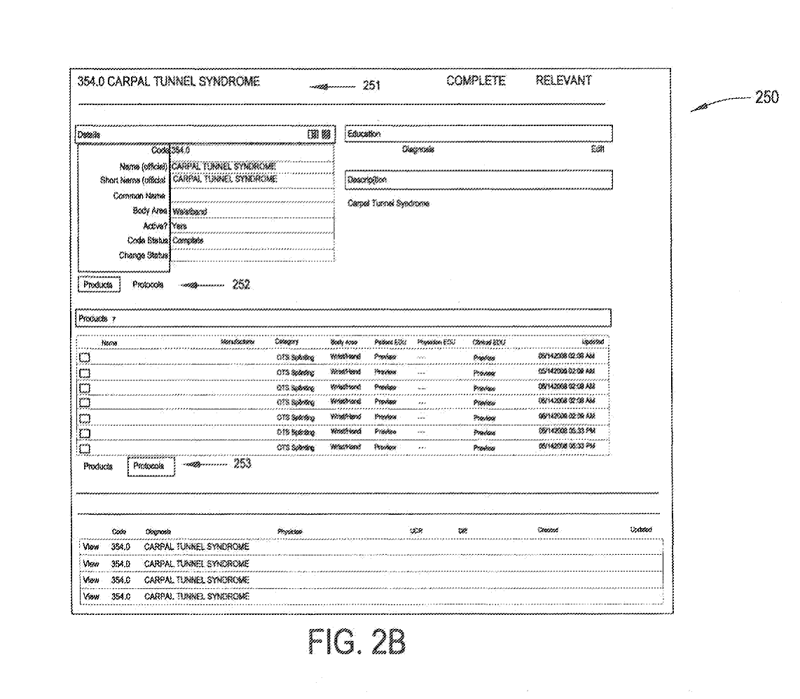

FIG. 1 illustrates example system architecture. In FIG. 1, a host system 20, for example a back end server or other suitable computing device, including appropriate hardware such as processor(s) and memory for storing executable program instructions, houses a healthcare and workflow management platform according to an embodiment. The platform of the host system 20 may comprise various system modules, including modules for interfacing with one or more remote device 10 (for example, phone, tablet, laptop computer, et cetera). In this regard, a remote device 10 may be utilized by a user (such as a healthcare provider, consultant, clinician, or physician, or by a public user, such as a patient) to access various functionalities offered by platform implemented on the host system 20.

Host system 20 includes a patient/public interface module 122 and a physician/healthcare provider interface module 124. Patient/public interface module 122 provides a publicly available interactive web site and/or downloadable application (app), for example as provisioned to a remote device 10. Healthcare provider/physician interface 124 provides interactive programs for indicating diagnoses, selecting protocols, ordering products/services, formulating patient agreements, billing and insurance handling, tracking (for example, revenue, customer satisfaction, and the like).

Host system 20 may also include a clinic device management system including an outside devices module for communicating with outside devices (such as wirelessly connected remote device 10), a service personnel module, and an outside providers module (for example, for communicating with hospitals, labs, et cetera), a fulfillment module, an inventory module, an intake module, a revenue module, an intelligent reporting module, and an application programming interface (API) connected to outside devices (for example, wireless network components, medical instruments, et cetera), or some suitable combination of the foregoing.

An example embodiment of a host system 20 for healthcare and workflow management thus includes a healthcare provider/physician interface 124. The healthcare provider/physician interface 124 handles information including, but not limited to: patient name, height, weight, date of birth, gender, contact information, insurance coverage, DME service history, and custom protocols based on physician. Within the healthcare provider/physician interface 124, the interface may manage physician information including, but not limited to: an ability to review patient symptoms and assign a diagnosis, and an ability to write a prescription, LMN, and/or CMN in host system 20. For clinicians and/or wellness points-of-contact (for example, a nurse practitioner, a physician's assistant, et cetera) the interface 124 may manage information including, but not limited to: clinician's organization, licensure (for example, PT, OT, MD, DO), availability of secured messaging with the host system service provider or other service providers, service or billing facilities, and associations of multiple patients by therapist or organization.

According to various embodiments, the system modules of the host system 20 may present a user of the host system 20 with one or more interfaces 122, 124. In one embodiment, interfaces 122, 124 may be presented to a user of the communications system 100 according to aspects illustrated herein. In general, the interfaces 122, 124 may be presented through an interactive computer screen to solicit information from and present information to a user in conjunction with accessing and using the various embodiments. In one implementation, the interfaces 122, 124 may be presented through access devices 10, for example, including personal computers running browser applications and having various input/output devices (for example, keyboard, mouse, touch screen, et cetera) for receiving user input.

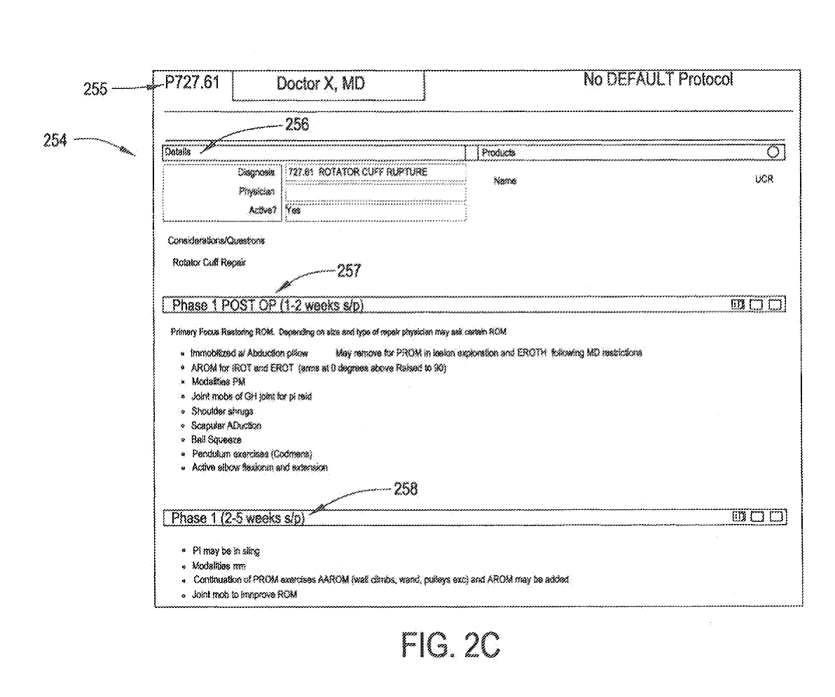

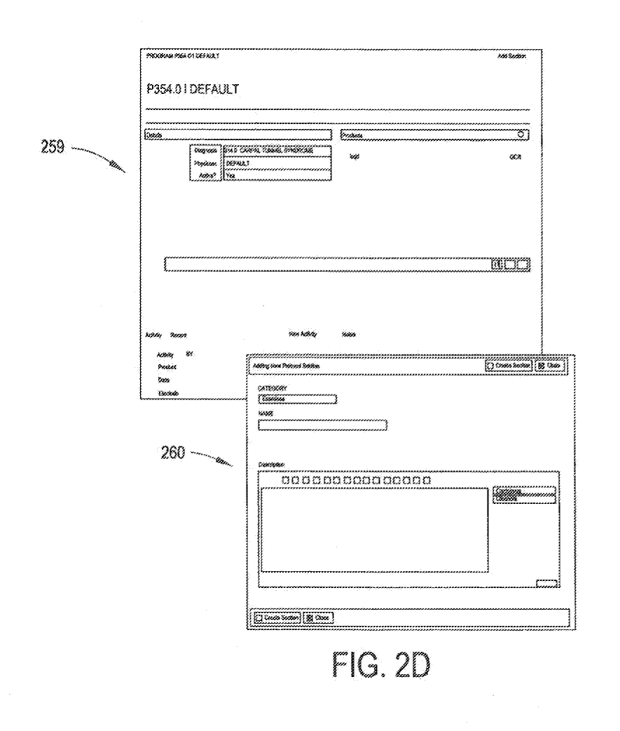

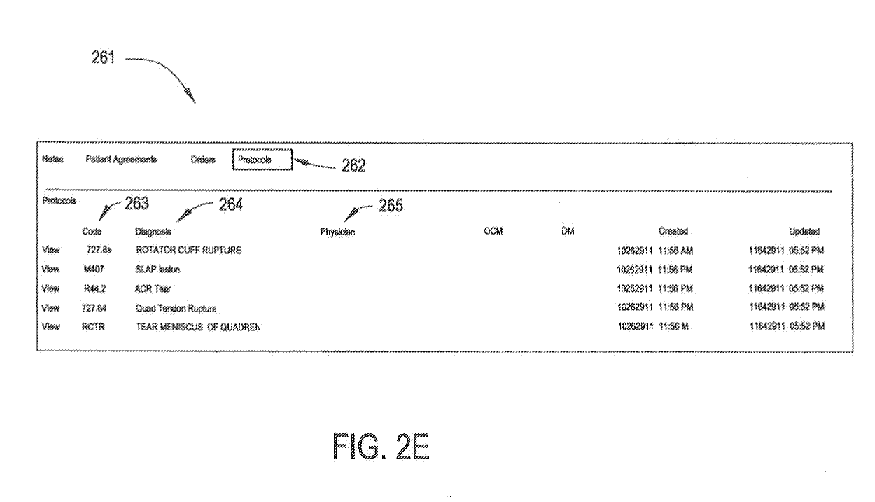

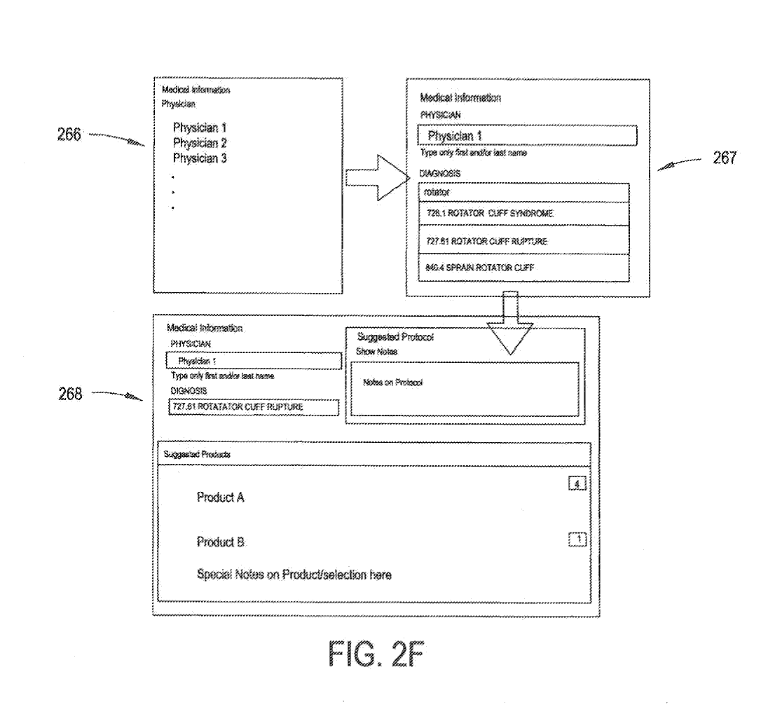

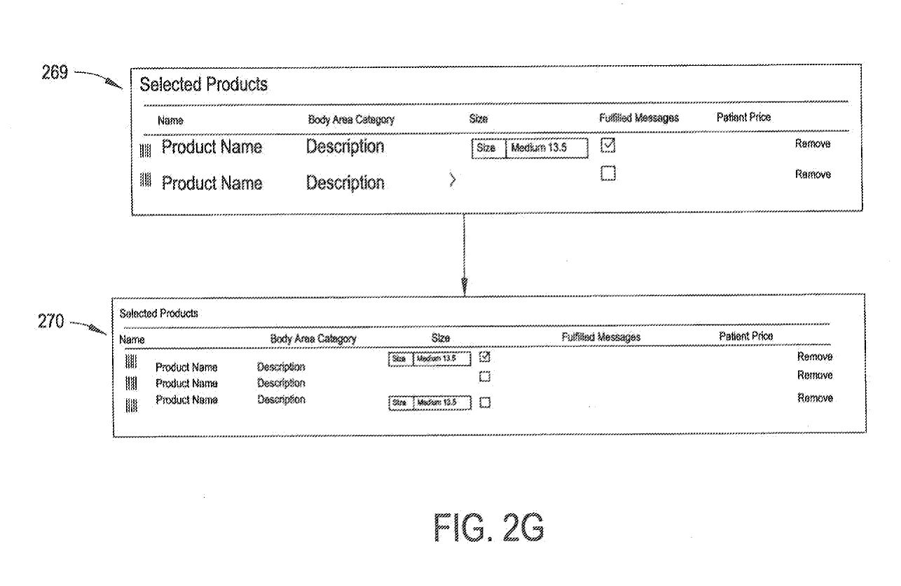

According to an example embodiment, as illustrated in FIG. 2A, once a patient's medical history has been stored in the system and subsequently identified, a user, which may a physician or a healthcare provider accessing system via interface 124, may select/input a diagnosis 210. Alternatively, the diagnosis may enter the system via a public interface, for example, via a patient conducting a virtual visit via interface 122. Responsive to a diagnosis being made available, the system 20 searches for corresponding treatment plan(s) 220 (which a user may customize via selecting an appropriate physician, protocol, products, services, and the like) and outputs the available treatment plan(s). As further described here, there may be more than one acceptable/approved treatment plan, and each such treatment plan may in turn include various products/services according to various protocols. An appropriate treatment plan may be selected and customized 230 in consultation with a patient and/or others, such as insurance providers.

In FIG. 2B an example screen view created by the system is illustrated. Here, a screen view 250, which may be provided in an application on a tablet computer or like device, matching a particular diagnosis (carpal tunnel syndrome in this case) is illustrated. The diagnosis may have an identifier/code 251, such as “354.0” in this case. The screen view 250 contains details such as diagnosis code, name, short name, common name, body area associated therewith, whether the protocol is active, if the protocol has changed (including a link to a change history (not shown for simplicity)), and a description, which may include links to educational information.

The screen view 250 may include a plurality of sub sections. For example, the diagnosis may have product(s) and protocol(s) associated therewith. Here for example a products view 252 is available for the diagnosis 251. There are seven products usable with this particular diagnosis. Similarly, there are illustrated protocols in the protocols view 253 of the screen view 250. There are four available protocols for the example diagnosis. The products view 252 may include information such as product name, manufacturer, category, body area, links to educational information, and timing data. Similarly, the protocols view 253 may include a code name (to match the diagnosis), a diagnosis name associated with the protocol, a physician that created the protocol, if any, a UCR (usual customary reasonable) amount and difference amount (relative to standard amount), and timing information (such as date created, et cetera).

In FIG. 2C an example screen view of a customized protocol 254 is illustrated. Here, a protocol number 255 (in this example, P272.61) identifies the protocol 254. The protocol 254 may be further identified by physician name, thus identifying the source of the protocol 254. The screen view of the customized protocol 254 may include a details portion 256, which may include information such as the diagnosis associated with the protocol, in this example “rotator cuff rupture”, whether the protocol is active, as well as associated products (none are listed for simplicity of illustration), and a considerations/questions area. The protocol may include phase areas if the protocol is segmented logically into different phases. Here the protocol includes two phase portions 257, 258. The phase portion(s) include a primary focus (here, restoring range of motion (ROM)), and the details of the treatment protocol for accomplishing the same. For example, the protocol may include recommendations for activity (or inactivity), for example immobilization, shoulder shrugs, scapular adduction, et cetera, as well as products (for example, pillow, sling) for use in the treatment.

FIG. 2D illustrates an example default protocol screen view 259. Similar to the custom protocol 254, the default protocol includes an identification code (P354.0—which matches the diagnosis carpal tunnel syndrome), a details portion including the diagnosis name (carpal tunnel syndrome in this example), an indication that it is a default protocol (for example a proprietary protocol that was not created by or assigned to any particular physician), and an indication if the protocol is active. The default protocol may include all or some of the same portions (for example, phase area(s)) as the custom protocol illustrated in FIG. 2C. Each protocol also may be modified, for example by way of an editing screen 260 as illustrated. Responsive to bringing up an editing screen (within an editable protocol), a user may enter a new protocol section or modify an existing one. Here, an editing screen 260 for adding a new protocol section (“exercises”) is illustrated. As illustrated, the editing screen 260 allows for text based input of information regarding a portion of the protocol, in this example the addition of an exercise that will be added to the protocol.

A user, such as a physician or healthcare provider may have personalized information stored in the system 20. For example, illustrated in FIG. 2E is a screen view 260 for a home page of a physician in the system that lists electronic notes, patient agreements, orders and protocols. A user, such as healthcare provider may use this interface to identify protocols associated with this physician. For example, by selecting a protocol tab 262, the view 261 displays protocols associated with the particular physician. In the view 261, protocols are listed, as identified by codes 263 for particular diagnoses 264 for the particular physician 265. The view 261 may include other information, for example pricing information and timing information (in this example, creation and modification times of the protocols).

After a diagnosis has been provided to a patient, either by way of in-person visit to a physician's office or clinic or via a virtual visit, an embodiment provides for facilitating formation of patient agreements, which formalize the treatment plan for each patient. Thus, a healthcare provider, for example a consultant or physician office staff member, may utilize a tool such as illustrated in FIG. 2F to facilitate efficient patient agreement formation.

As illustrated in FIG. 2F, a user (such as a healthcare provider, for example a consultant or physician office staff member) may, in consultation with a patient, select, for example via drop down menu 266, a physician that provided the diagnosis to the patient. Responsive to finding the appropriate physician, the system provides information regarding diagnoses for the physician, for example in a second drop down menu selector 267. Once the patient has identified the physician and the diagnosis has been selected, the system provides the suggested protocol the particular physician prefers be followed for the given diagnosis. Thus, the system provides information regarding the suggested protocol in a suggested protocol view 268. The suggested protocol view 268 may include a variety of information, such as suggested products, their availability, special notes modifying the protocol or informing on a preferred implementation of the protocol, and a link to the actual protocol view itself (for example, a link to the protocol for rotator cuff rupture in this example).

The system 20 also facilitates selection and ordering of products and/or services. Using products as an example, an embodiment facilitates selection of products for inclusion in a treatment plan, as illustrated in FIG. 2G. Here, a first screen view 269 illustrates two products suggested for use in a protocol. The user may select among the products for inclusion. In addition, a user may add additional products to the treatment plan, as illustrated in updated screen view 270. Thus, the system 20 facilitates listing and selection of available products, for example as included in a recommended protocol, for use in a given treatment plan. The selected products are included in the patient's treatment plan and eventually in the patient's order, as further described herein.

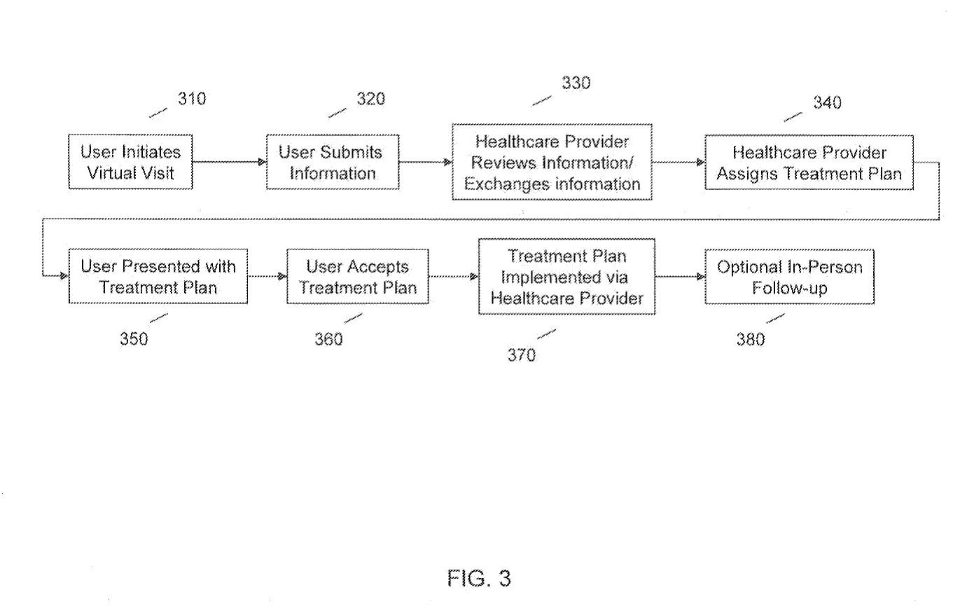

According to an example embodiment as illustrated in FIG. 3, a user may create a virtual or web-based visit 310, for example via interface 122, with a physician as part of the healthcare and workflow management system. This virtual visit may include exchange of information, for example a user may submit information 320 in the form of a recorded Q&A chat session, a recorded video-conferencing session, and submission of photos, medical images, or medical test results, facilitated through the use of mobile device(s), computer(s), or kiosk(s) executed in either a live or recorded format. Once pertinent information has be conveyed and transmitted, the physician performs a review of the information 330. During this process, the physician may assign a treatment plan and fill out any necessary prescriptions 340, or the physician may request additional information. If no additional information is required, the treatment plan and/or prescriptions are submitted to the patient 350. If the patient accepts the plan 360, clinicians or other qualified practitioners may be notified to service the patient 370. Options for following up during and after the treatment plan may also be facilitated 380.

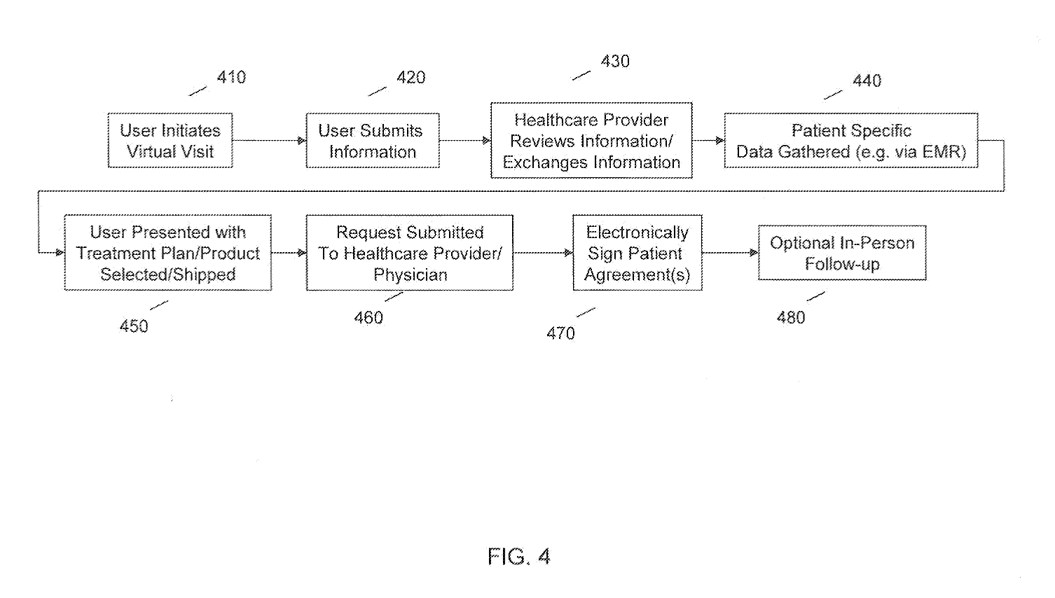

According to another example embodiment as illustrated in FIG. 4, a user may create a visit 410 with a clinical specialist as part of the healthcare and workflow management. Like the virtual or web-based visit 310 with a physician, this virtual visit may include exchange of information, for example a user may submit information 420 via a recorded Q&A chat session, a recorded video-conferencing session, and submission of photos, medical images, or medical test results, facilitated through the use of mobile device(s), computer(s), or kiosk(s), executed in either a live or recorded format. Information from the virtual visit may be evaluated by a healthcare provider or other service providers 430, where the healthcare provider provides instructions to the patient, answers questions, directs patients to perform measurements, et cetera. During this process data may be automatically retrieved from a practice management database, insurance company and/or electronic medical record (EMR) 440.

After the healthcare provider has retrieved the patient's data during the visit, a treatment plan may be developed and/or a product may be dispensed at a clinic or shipped/delivered to the patient 450. In another embodiment, the treatment plan may be automatically assigned based upon patient demographics, doctor protocols, and/or diagnoses. A request may also be submitted to a physician to electronically sign a prescription, LMN, and/or CMN 460. Patients' agreement(s) may also be electronically signed by the patient using the system 470. Additionally, options for following up with the physician during and after the treatment plan may also be facilitated 480.

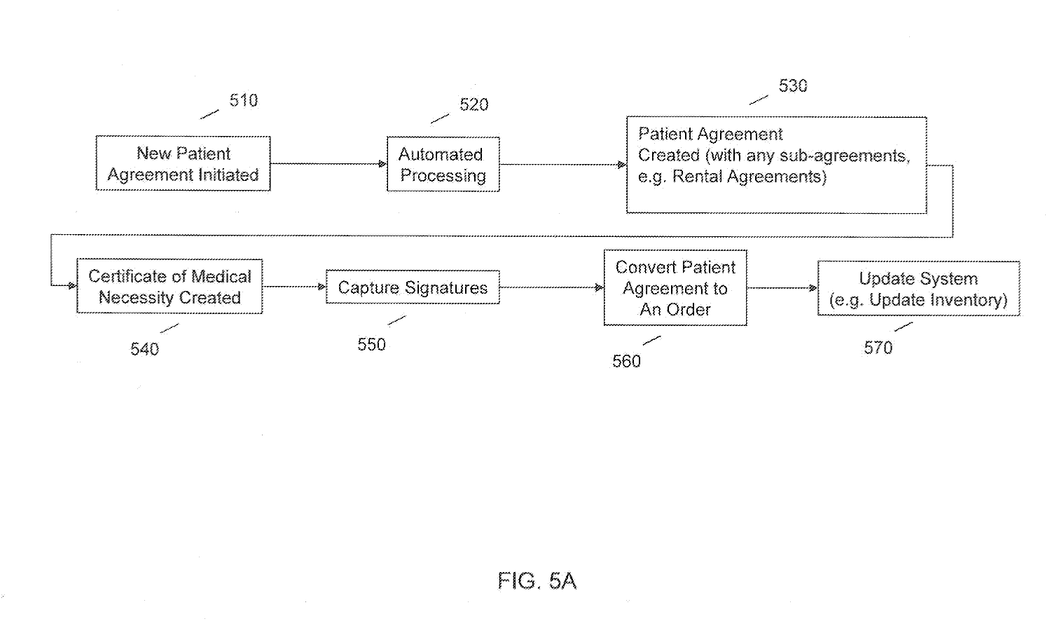

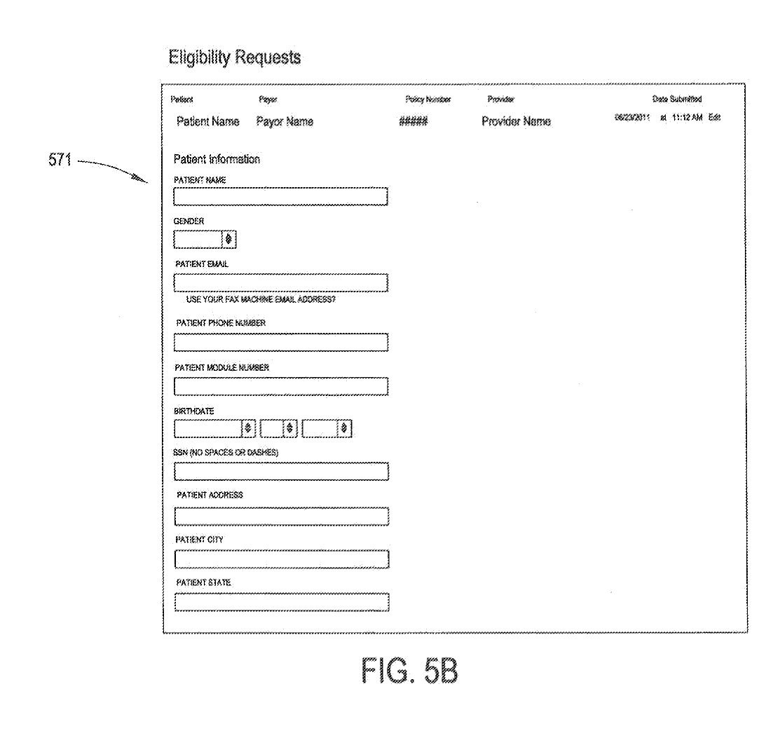

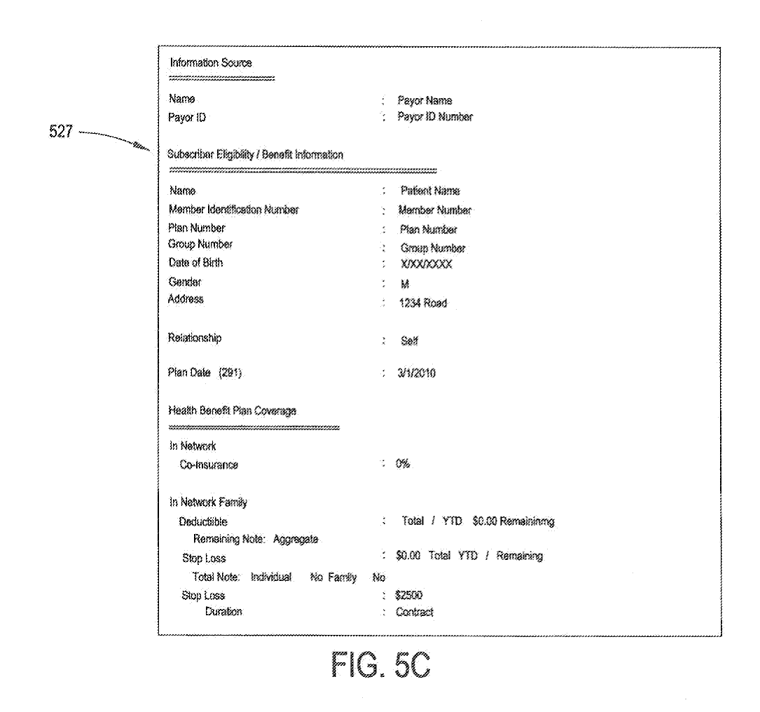

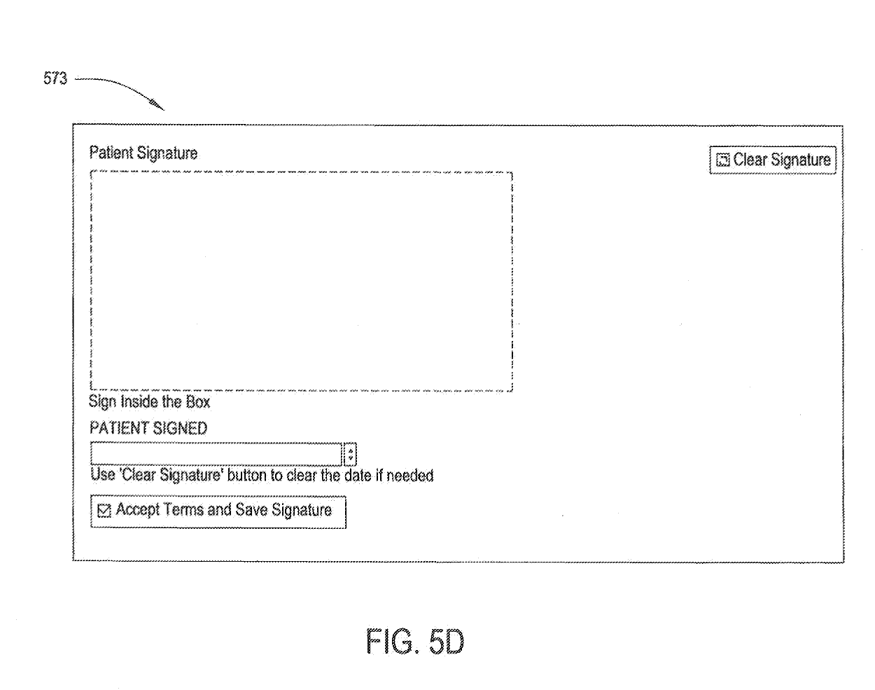

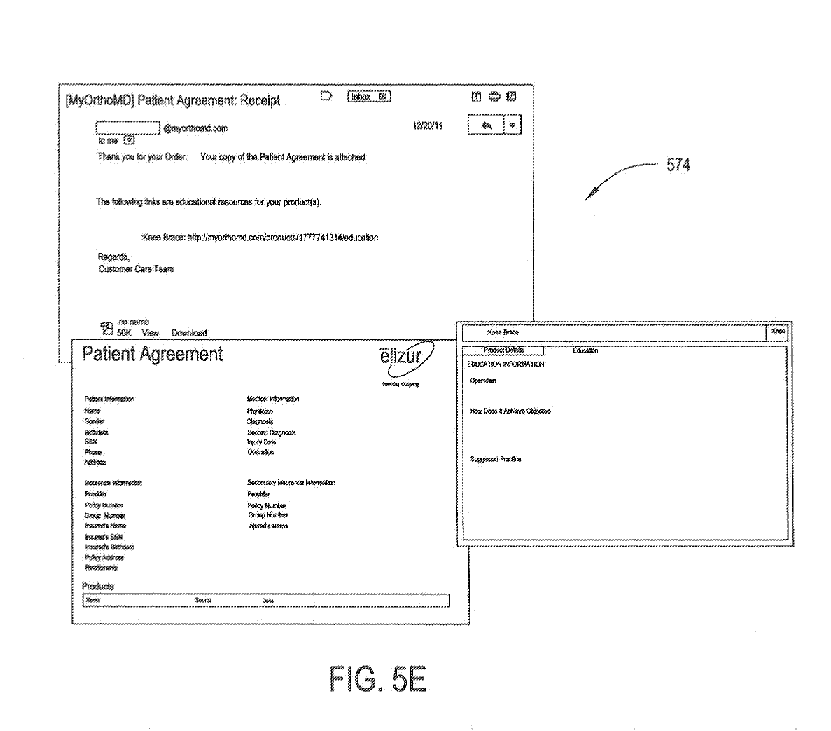

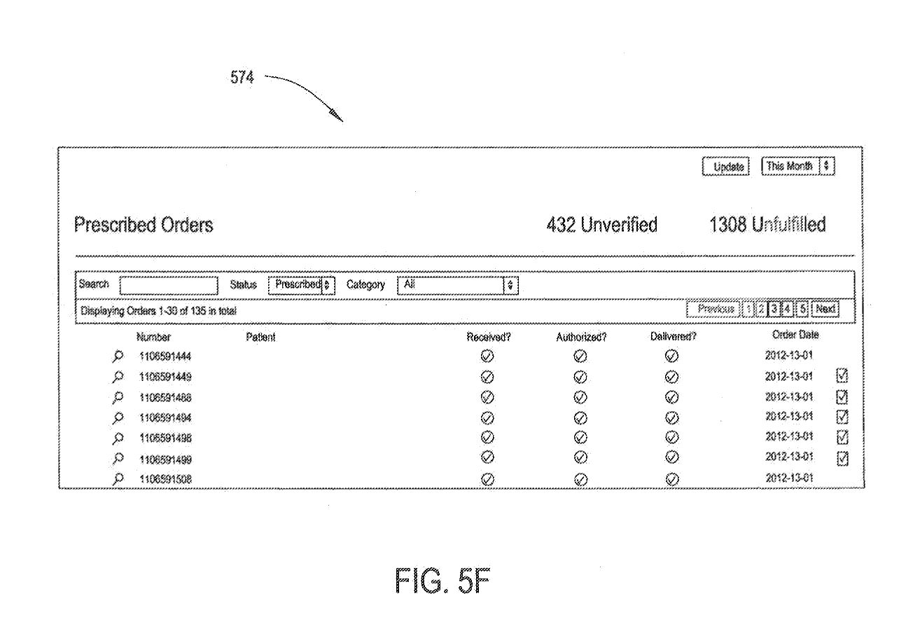

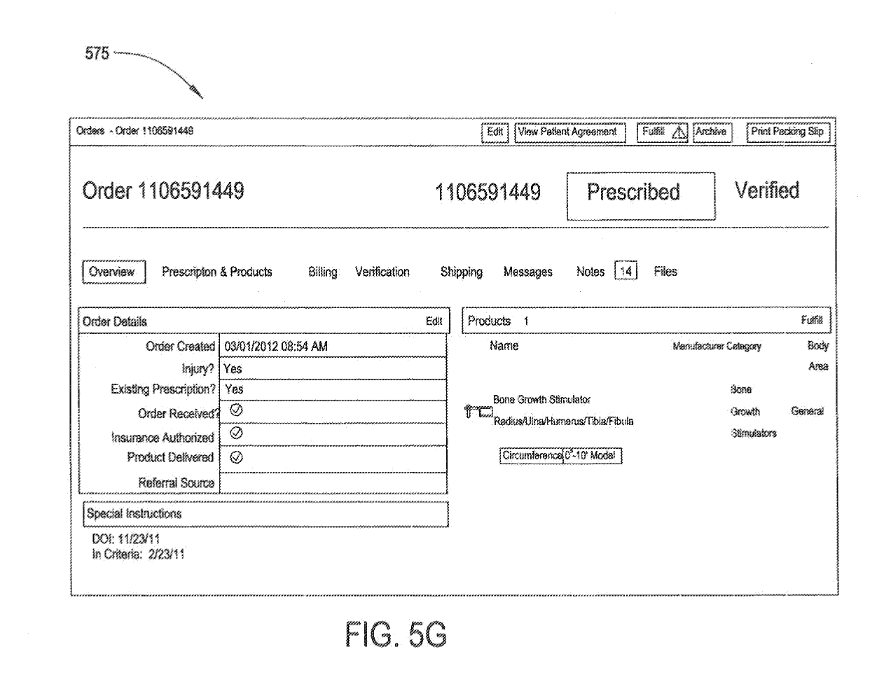

According to an example embodiment, illustrated in FIG. 5A, a process may be automated to increase accuracy and efficiency of forming and implementing a patient agreement, and may be incorporated with the healthcare and workflow management system. As illustrated in FIG. 5A, when a user initiates creation of a new patient agreement 510 (for example, when a patient requests products and/or services responsive to a diagnosis), an automated process may be employed 520 to check for insurance eligibility, link a diagnosis to a patient agreement, link a product to an inventory, and/or automatically populate a patient's demographics and other information from the payor or practice EMR/management system.

Illustrated in FIG. 5B, an embodiment facilitates automated (electronic) eligibility inquiries/requests. An eligibility request screen view 571 is provided by an embodiment to allow eligibility for a particular product, service, or combination thereof to be checked for eligibility at a payor, for example the patient's insurer. An embodiment includes editable fields, which may be auto-populated by accessing a patient ERM, for identifying the patient by name, gender, email, phone numbers, birth dates, SSN, address, city and state, and/or other information. The eligibility request is submitted to the payor, along with any pertinent details regarding the treatment, products, services, et cetera being requested. The submission may be made electronically.

FIG. 5C illustrates an example response 572 of a payer to an eligibility request. The response indicates identifying information for both the payor and patient (including policy specific details regarding coverage). The response of the payor 572 allows the system to accurately check for payment coverage for particular treatments, products and/or services.

In addition to automated processing 520, a new patient agreement may be created 530 on the basis of the selected treatment plan (including products and/or services), and a certificate of medical necessity may be created 540, along with electronically capturing the necessary signatures to create a patient agreement. FIG. 5D illustrates an example screen view 573 for capturing a patient's signature, which may include provisioning of legal information (not illustrated). The physician's signature for prescription and/or treatment plans may also similarly be captured 550. Once the signatures have been captured, copies of the agreement(s) and documents then may be saved and sent to the patient and relevant clinic electronically, for example, by email (574 as illustrated in FIG. 5E), by facsimile, and/or in print. Lastly the patient agreement may be converted into an order 560 such that the system may process the order and update 570 (for example, order a product from inventory according to the patient agreement, and update the inventory database).

Once a patient agreement, including selected products, services and the like has been formalized, the patient agreement may be converted into an order. An order may include requests for products and/or services called for in the patient agreement. FIG. 5F illustrates an example of an order screen view 574. The order screen view includes information regarding pending orders in the system 20. The order screen view 574 may be accessed by a user that in some way needs information regarding a particular order. For example, a consultant or physician office staff person may access order screen view 574 to track or update information regarding a pending order. When in order screen view 574, a user may select a particular order to view more information regarding the same. The order screen view 574 itself may include some quick reference information in various fields, for example a received field (indicating if the order has been received by a party that will fill the order), an authorized field (indicating if the order has been authorized), a delivered field (indicating if the products/services in the order have been delivered), and date and timing information fields.

Responsive to a user selecting a particular order from within the order screen view 574, the system 20 brings up an order detail view 575, illustrated in FIG. 5G. The order detail view 575 includes order information pertinent to the particular order, such as an order details area and a product area. In the order details area is listed information including for example the order creation date, if there was an injury, if there is an existing prescription, if the order has been received, if the insurance has been authorized for the order (or part thereof), if the product has been delivered, and a referral source (for example, physician name), if any. The product area may include product related information such as a name of the product, a manufacturer of the product, a category for the product, and the like. A user may access certain functionalities from within the order details view 574, such as an edit tab (allowing the order to be edited, updated or otherwise modified), a view patient agreement tab (allowing the user to bring up the patient agreement from which the order was derived), a fulfill tab (allowing the user to provide information regarding order fulfillment status), an archive tab (allowing the user to access archived information regarding the order, such as a change history), and a print packing slip tab (allowing the user to view and print a packing slip for a physical product, if any).

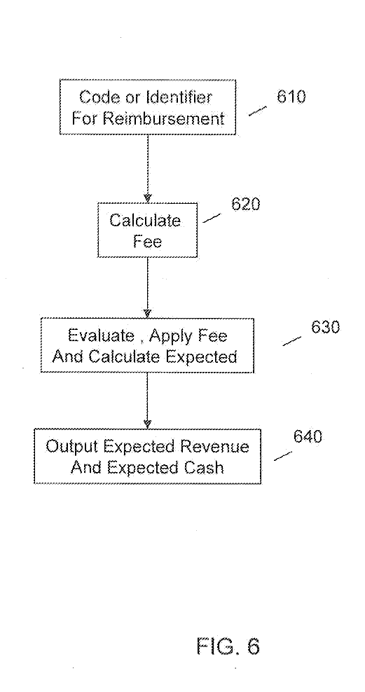

According to another example embodiment as illustrated in FIG. 6, a fee schedule generation process for services rendered may be incorporated with the healthcare and workflow management system 100, as for example accessed via interface 124. An identifier (for example, HCPCS (healthcare common procedure coding system) Code or other identifiers) for reimbursement may be assigned 610. Several different methods of determining the appropriate fee may be employed 620. These include calculating an average based on other insurers' past charges or reimbursements, calculating a fee based on past insurers' explanation of benefits (EOB), or manually setting and entering a fee. An embodiment automates this process by accessing historical billing information, as stored in proprietary data store, such as housed in database 214. Once the fee has been determined, it may be evaluated and applied to calculate expected values, such as expected cash and revenue 630. This may include for example evaluation under a UCR standard, and once the UCR standard has been applied and met, expected revenue and expected cash may be calculated (for example, as expected by the clinic or clinical specialist) and output 640. In an example embodiment, the calculations may be automatically performed by the system and presented to the clinic and/or clinical specialist.

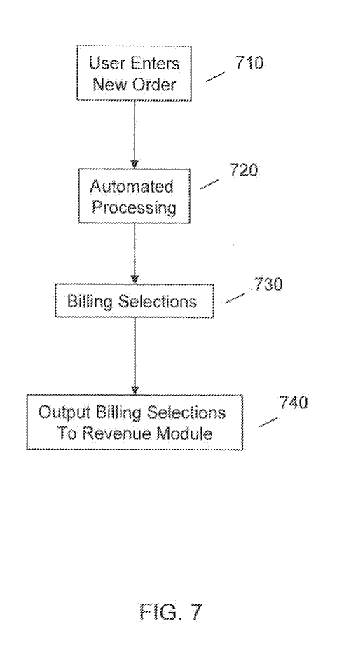

According to an example embodiment as illustrated in FIG. 7, an intake management process may be incorporated with the healthcare and workflow management system 100. The intake process is initiated when a user enters a new order 710. Automated processing 720 may be employed in connection with the new order in preparation for completing the order and billing. The automated processing 720 may include, but is not limited to, checking for insurance eligibility, linking relevant physician(s) to the order, linking a diagnosis to the order, linking required products to an inventory, and/or automatically populating a patient's demographics and other information from payor or practice EMR/management systems. Once automated processing 720 services have been completed, a biller, contractor, payor and plan may be selected as part of billings selections 730. A fee schedule may also be assigned as part of this process. During this process, the status of paperwork may also be maintained. Once the automated processing 720 and the billing selections 730 have been made, the resultant processed order information may be sent to a billing/revenue module for further processing.

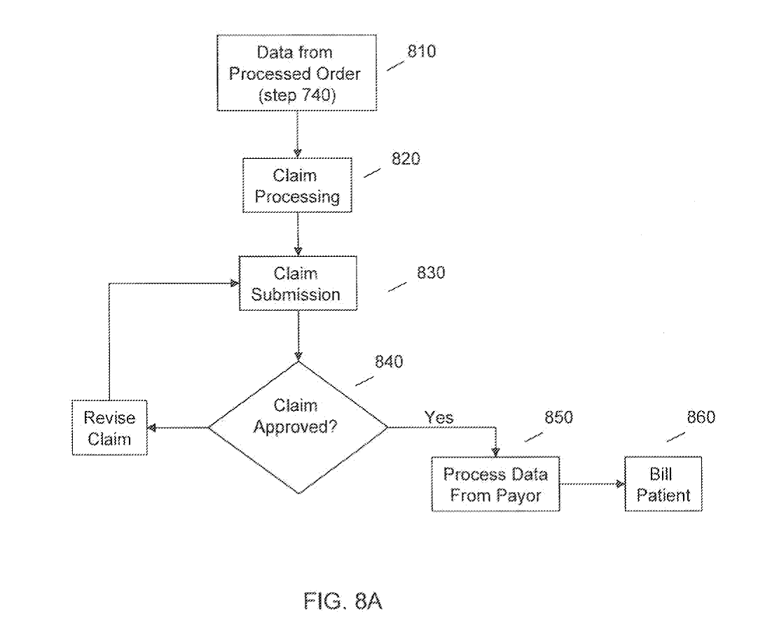

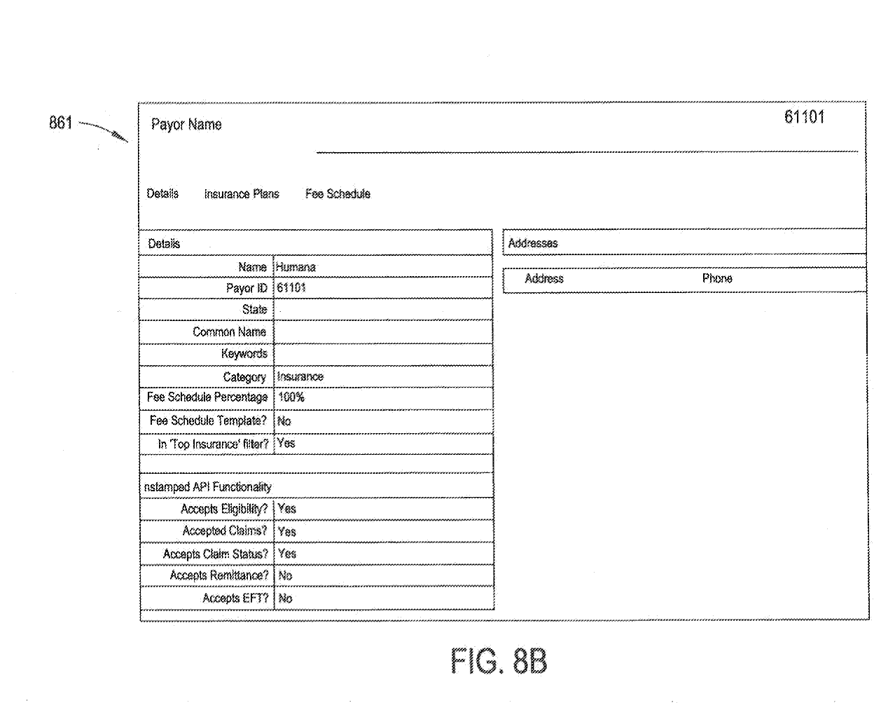

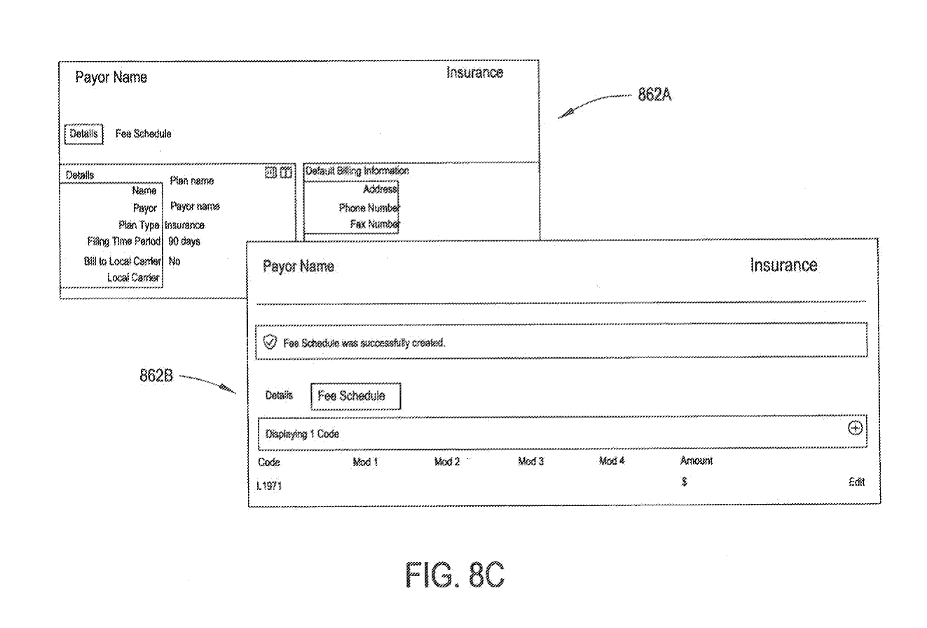

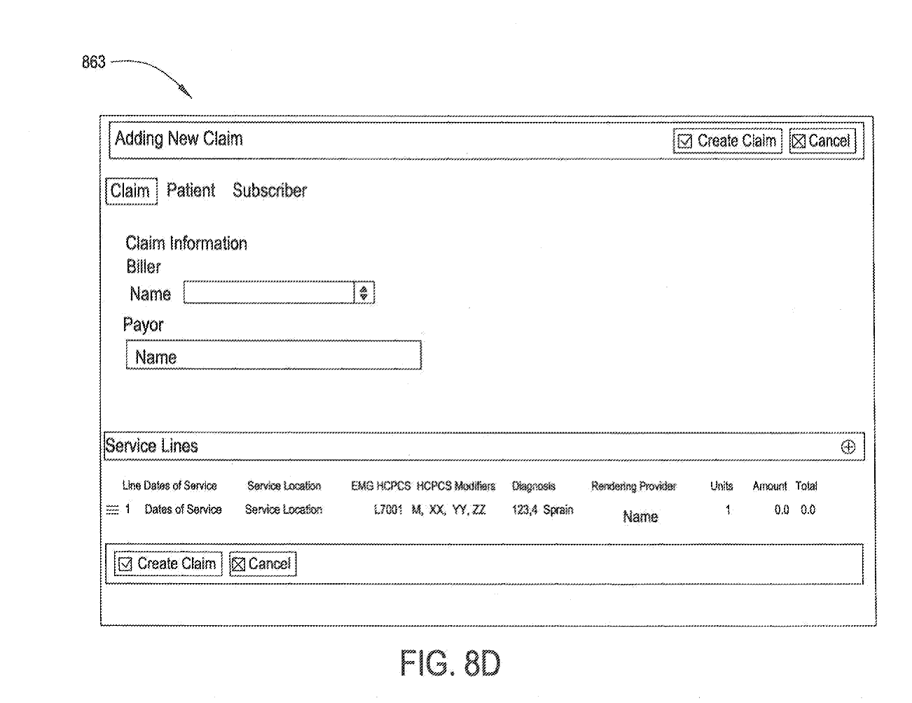

As illustrated in FIG. 8A, within a billing/revenue module of healthcare and workflow management system 100, data associated with the order is retrieved 810. The data from the order is used in claim processing 820. The claim processing may include checking eligibility of the patient and/or treatment plan (or components thereof) against the payor requirements. The HCPCS code may be determined prior to the claim being scrubbed, as it is assigned at the product level. This in turn gathers data from the payor. After the claim is scrubbed, the diagnosis may be checked. Once analyzed, the healthcare and workflow management system 100 may calculate and provide a probability that the claim will be paid (prior to submission of the claim at 830) and may additionally provide alternative coding suggestions to improve the probability of the claim being paid. After the claim has been scrubbed, a “1500 Form” may be generated from the available data, and submitted 830 to a clearing house either directly through the payors, electronic data interchange (EDI), or printed for mailing. On the payors' end, the submitted claim is reviewed and is either accepted, denied, or returned with a request for additional information, as determined at 840. If the payor accepts, data from the payor is processed 850, for example an explanation of benefits (EOB) is retrieved and applied to the claim. A payment is then posted and a bill is generated to the patient 860.

Illustrated in FIG. 8B is an example of a payor profile view 861. The payor profile view 861 presents organized information about a particular payor of claims made, for example a health insurance company. Payor profile view 861 allows a user to quickly ascertain information regarding a payor. Information regarding a payor, such as the information provided by payor profile view 861, may be used to implement pattern based learning to generate rules per payor or plan, or suitable combinations thereof. These pattern based rules may inform the user in preparing a claim for the payor, for example via suggesting appropriate coding for particular treatment plans' products and/or services, historically approved by the payor.

The system 20 provides for the ability to manage payors and to interface by electronically checking for eligibility, submitting claims, checking claim status, providing remittance, and EFT. Payors can be set up to mirror another payor's fee schedule, or as a percentage of another payor's fee schedule. Multiple forms of contact can be stored in the record of FIG. 8B and linked into other areas/modules of the platform/system 20, including patient agreements, patient orders, and claims.

For example, illustrated in FIG. 8C, fee schedules may be stored and displayed by payor or insurance plan 862A, allowing for a customization or modification of fee schedules and medical policies per HCPCS or procedure code, as illustrated in 862B. The data is pulled into a patient agreement and/or order (automatically entered into these area screens) so that relevant payor related fee information can be collected and provided at the point of care (that is, at the time that the patient and healthcare provider form the patient agreement and/or order). Among other advantages, the ability to access and utilize payor specific information during this phase of care increases the likelihood of submitting a proper claim to the payor/insurer, including using suggested coding derived from pattern based learning, as described herein. Certain (insurance) plans may trigger special attention and consideration for a variety of reimbursement factors, including the type of claim submission, the time period for authorization, and support for third party administrators.

As illustrated in FIG. 8D, a claim addition tool 863 allows orders to be converted to electronic claims efficiently. A probability of being paid (claim submission being honored) may be calculated against previous similar claims (historical information) acceptance/rejection rates, timing, et cetera. Acceptance/rejection and reimbursement trends against claims for various providers may thus be collected. Factors impacting acceptance and timing of claim payments include HCPCS code, diagnosis, payor, insurance plan, and available documentation. By tracking such information, the system provides a proprietary database, for example database 214, for providing predictions of claim acceptability and offering alternatives/modifications to claim submissions to help ensure claims are acceptable to the payor.

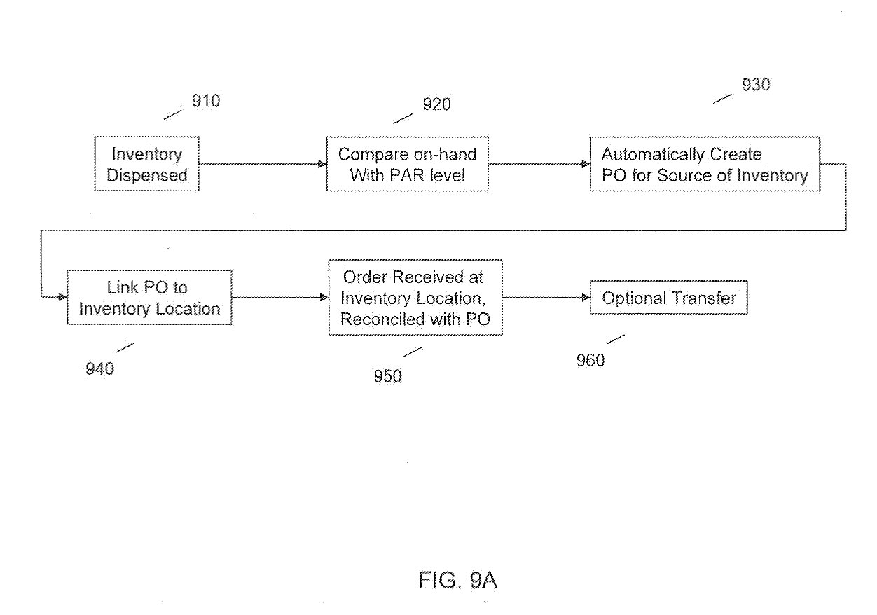

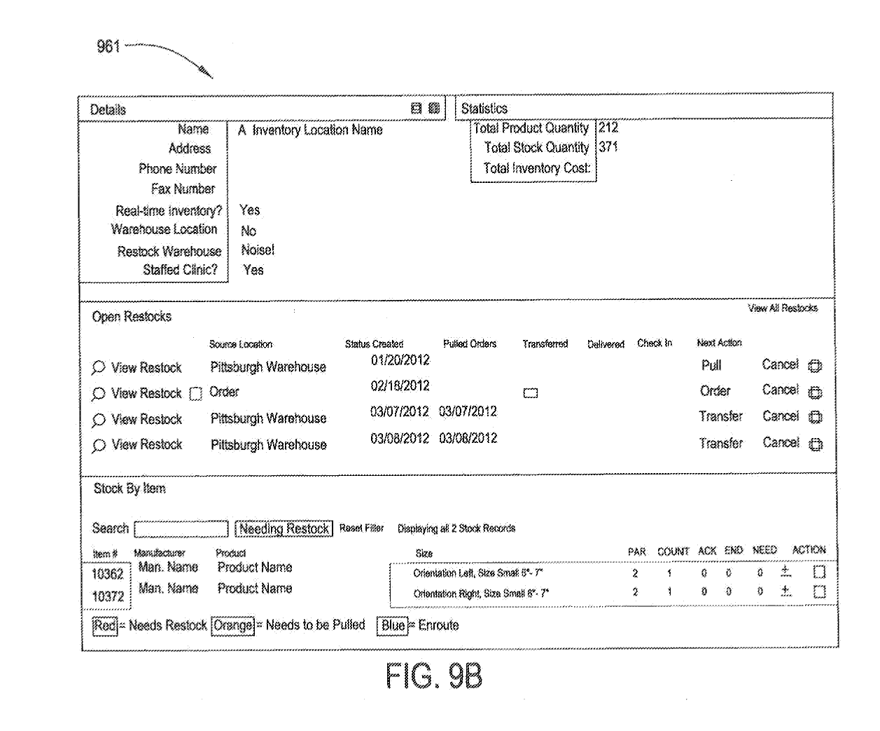

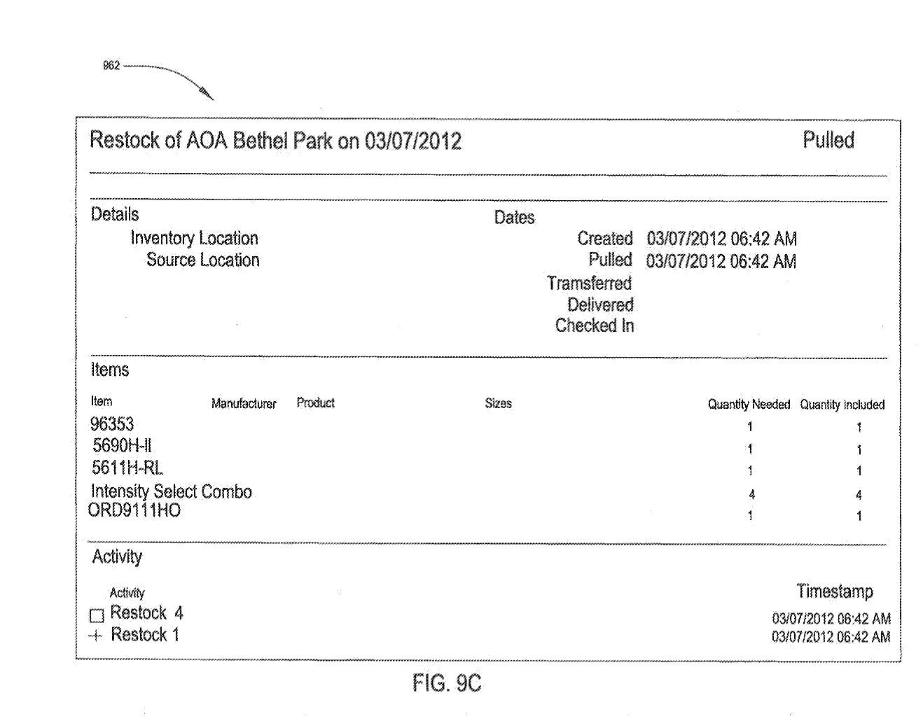

According to an example embodiment as illustrated in FIG. 9A, an inventory processing module may be incorporated with the healthcare and workflow management system 100. When an order is created and processed, a product may be dispensed from an inventory location 910. The system then compares the inventory on hand with a predetermined periodic automatic replenishment (PAR) level 920. If necessary a purchase order (PO) may be created and submitted to the pertinent source/manufacturer 930. Additionally the PO may be linked to the inventory location 940. Once the order has been received at the inventory location, it may be reconciled with the PO 950. The inventory process may also provide the option of transferring the order to another inventory location 960. The system 100 can further track returned products and/or services and may automatically rank product and/or service information including, for example, patient ease of use, returns, quality, clinical efficacy, clinician satisfaction, et cetera.

For example, illustrated in FIG. 9B is an inventory view 961 provided by the system 20 for reviewing and managing medical product inventory of a particular location. The inventory view 961 may include details and statistics areas regarding a medical products (or services) available at the location, such as a brace or a sling associated with a protocol of a treatment plan.

The details area in the inventory view 961 may provide inventory location details, such as warehouse name and location information. The statistics area of inventory view 961 may include statistical information regarding the quantity/availability and cost/value of products/services at the warehouse. An open restocks area of the inventory view 961 may provide a summary of current restock actions regarding the product(s) being restocked (for example, a brace that was sold, deducted from inventory, and reordered) with respect to a source location for the product, a date and time of creation of a restock action, as well as current and future status information (transferred, delivered, checked in, and/or next action (pull, transfer, order, et cetera)).

FIG. 9C provides an example view of an open restock ticket 962 provided responsive to a user opening an open restock item in view 961. In FIG. 9C, an open restock ticket 962 is provided by the system in response to a user indicating to the system that item(s) for a given inventory location need restocked or their inventory status is to otherwise be modified within the system. Thus, the restock ticket 962 provides means to order an item for restock and track the restock progress within the system 20.

The inventory view 961 may also include a stock by item area that provides information regarding products categorized by status. For example, in the inventory view 961 illustrated in FIG. 9B, a user has searched for items indicated in the system as needing to be restocked. This inventory view 961 provides a listing of products (product name, manufacturer name, item number) and their status regarding restocking. For example, two items are listed in the stock by item view filtered for items needing restocking. In the example of FIG. 9B, the items' PAR is 2, there is a count (available) of 1, and no restocking order has been acknowledged or entered, thus the view indicates that one of each product is needed (a restock order is needed for each product). Such tracking may advantageous for managing medical product/device inventories, and may be extended to tracking and managing service provider time or availability as a commodity.

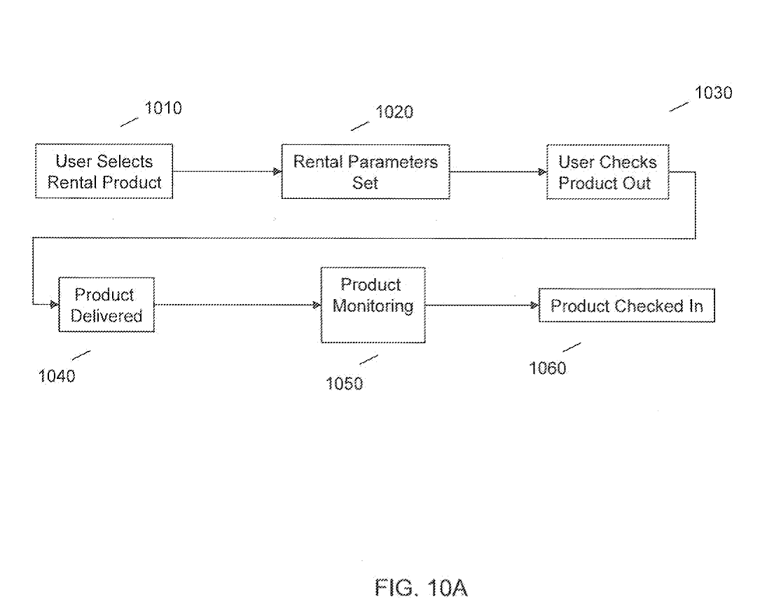

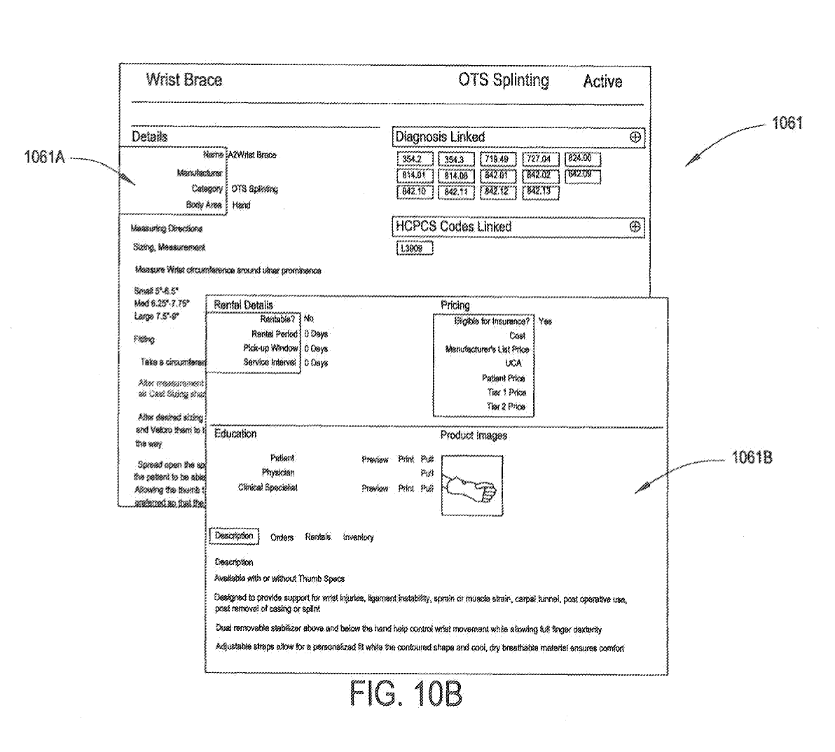

According to another example embodiment as illustrated in FIG. 10A, a rental management process may be incorporated with the healthcare and workflow management system 100. Once a treatment plan has been determined and a user, such as a healthcare provider, selects a rentable product 1010, parameters for standard rental, maintenance, or other factors may be set 1020. An asset may be created and the product(s) may be assigned a store serial number, asset number, or other unique identification. The user then checks out the product 1030 and the user delivers the product to a patient. Each of the checked out product(s) are associated with a specific patient according to the above identification means. The system then monitors the product for a subsequent pick-up date and/or maintenance needs 1050. When the rental period has concluded, the user picks up the product(s) from the patient and checks the product back in 1060. The product may then be serviced, maintained, or discarded if beyond repair.

Illustrated in FIG. 10B is an example product view 1061 provided by the system 20. In the example, a wrist brace is the example product provided in a basic view 1061A. In the basic view 1061A, details regarding the product are provided, such as the name of the product, the category of the product, measuring directions, and the like. Importantly, linked diagnoses and HCPCS codes associated with the product, in this case a particular wrist brace, are provided in basic view 1061A. This links the product to particular diagnoses. For example, when a physician makes a particular diagnosis, such as carpal tunnel syndrome, the diagnosis code may be used to match with a specific product for inclusion in a protocol. Moreover, similar to other (non-rental) products, the system can track internally information about the rental product, such as inventory status (checked out, checked in, and the like), pricing, coverage eligibility by payor/insurer, and the like. Product view 1061 also provides a rental details view 1061B that includes information regarding if the product is rentable (in this case, the wrist brace is not rentable but is only offered for sale), what the rental period is, pick up information, service information, pricing information, including insurance eligibility, educational information and even product images. Additionally, rental details view 1061B may include links to other system information, such as orders related to this product (searchable data base indicating patient orders for the product), rentals information (indicating currently rented products of this kind), and inventory (availability of the product for rent or sale).

FIG. 10C illustrates an example view of a device status screen. An embodiment allows a user to search for a particular rental product using a search interface 1062A. Once a product has been identified using the search interface 1062A, a user may select a product from the search results list in the interface 1062A to navigate to a detailed rental product status view 1062B. In the detailed view 1062B, product details, including identification details and location details are available for user review and editing.

According to another example embodiment as illustrated in FIG. 11, a customer satisfaction process may be incorporated with the healthcare and workflow management system 100. Here the system provides a means for customers to complete surveys via the phone, Internet, postcards, email, text, tablet, et cetera 1110. Once the customer surveys have been received, the system 100 finds the corresponding order based on the customer's input 1120. The customer and the survey are then linked to that particular order 1130 and the system determines and presents satisfaction scores for that particular order and provides links to appropriate system modules 1140. The customer in this regard may also be a physician or other healthcare provider that provides appropriate responses for tracking this type of customer (or more generally a user) satisfaction with a particular aspect or aspects of the system.

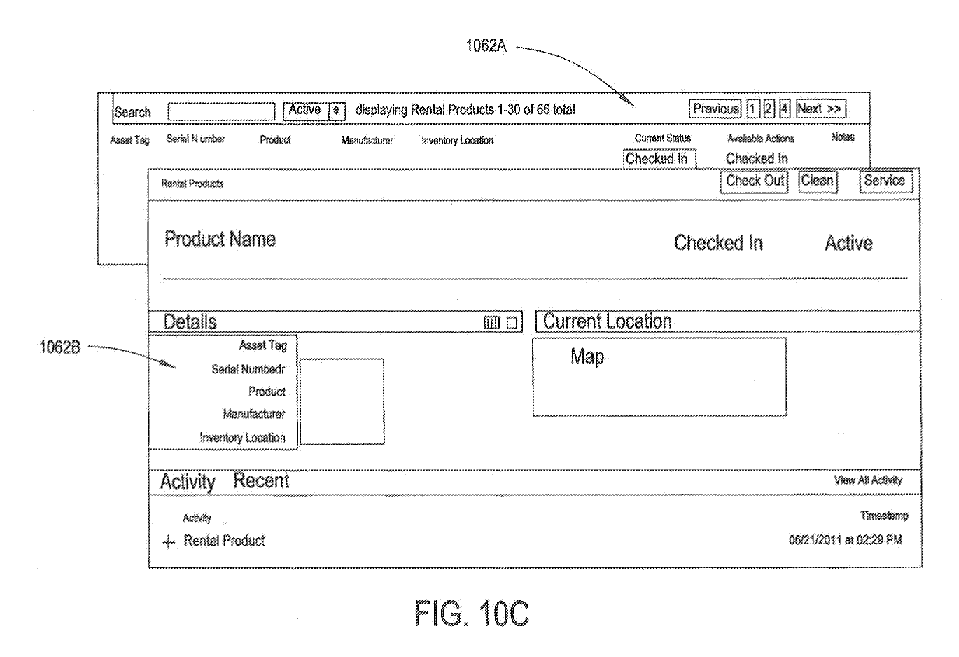

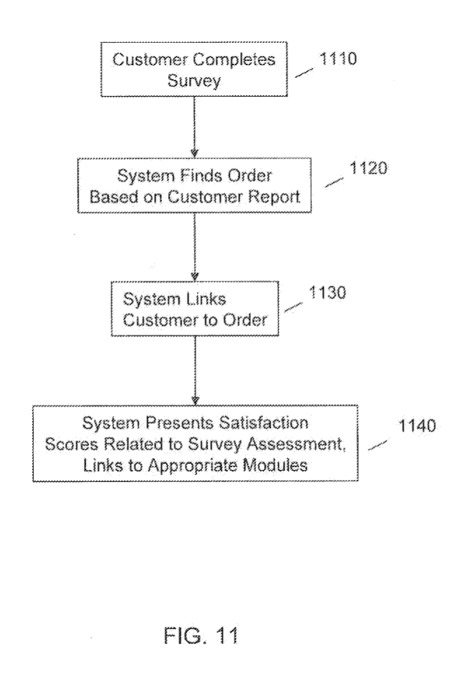

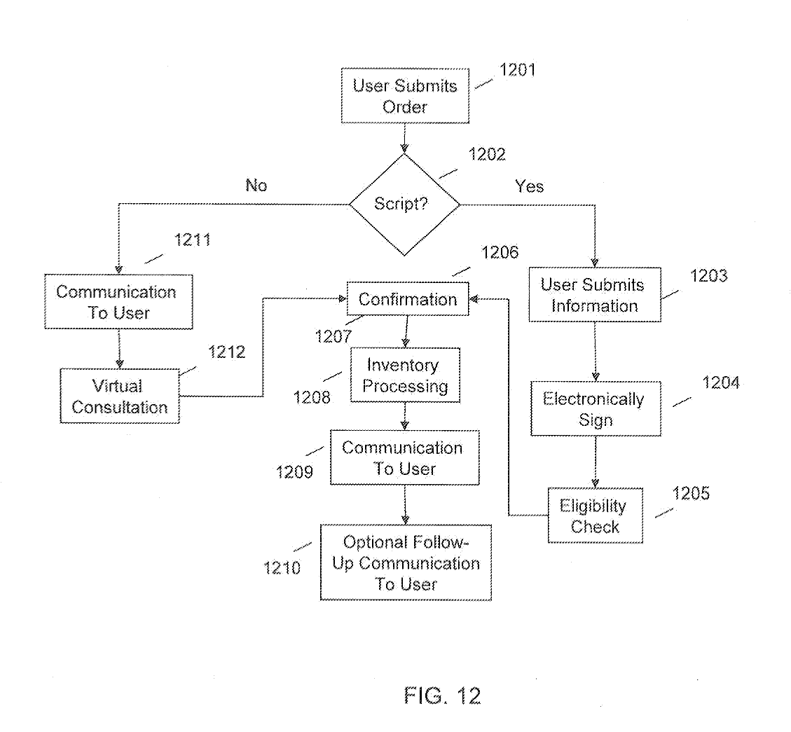

According to another example embodiment as illustrated in FIG. 12, a public interface 122 such as a web site or downloadable application may be incorporated with the healthcare and workflow management system 100. With this publically available interface, clinicians and patients can access and fulfill prescriptions for products and/or services, such as DME orders, online using a computer or other mobile device, for example one or more remote devices such as remote device 10. Additionally, an accessible interface 122, such as a website, enables patients to consult with a clinical specialist or a physician (healthcare provider) if they do not have an existing prescription.

For example, using a public web site or a downloaded application, a user submits an order 1201 and is prompted for a prescription 1202. If no prescription is available, the user may be directed 1211 to a virtual consultation 1212 (for example, with a physician, clinical specialist, or other healthcare provider or representative). Here the user is connected with a professional that will provide the user with a virtual consultation 1212, for example consistent with the virtual consultations or visits described herein. In addition to or in lieu of a connection with a professional that will provide the user with a virtual consultation 1212, the virtual consultation 1212 may also include a diagnosis and/or treatment assigned by the system based on the inputs from the potential patient/customer and referencing the database of standard care with an accompanying treatment plan that may include products, services such as therapy or behavioral recommendations, medicines or other modalities. This assignment may be reviewed and approved by a healthcare provider.

If the user already has a prescription available when prompted at 1202, the user proceeds to step 1203 where they may select the product required, input information 1204, for example demographics, insurance information (if available), shipping address, and other demographic information necessary. Also in step 1204, the user may electronically sign for the order and all the information may be electronically stored. Insurance coverage and eligibility on the order may also be automatically checked and verified 1206 in a confirmation process.

Once the order has been received, a confirmation may be sent back to the user. The items (products, services, et cetera) selected in the order are checked against the current inventory for availability and if it is not available it may be custom ordered 1208. An entity overseeing the system, or other service providers, may then perform fulfillment post-processing services. The inventory is then updated to reflect a deduction in the ordered products and a final confirmation or alert may be sent to the user 1209 and it may be in the form of an email, text message, phone call (automated or live), or other similar message alerts, for example. Once the order has been completed, a survey in the form of a phone call, email, or other survey means may be employed to track user satisfaction 1210. Survey responses received then may be stored and can be later reviewed to improve recommendations given during the virtual consultations and/or improve the overall user experience with the website interface.

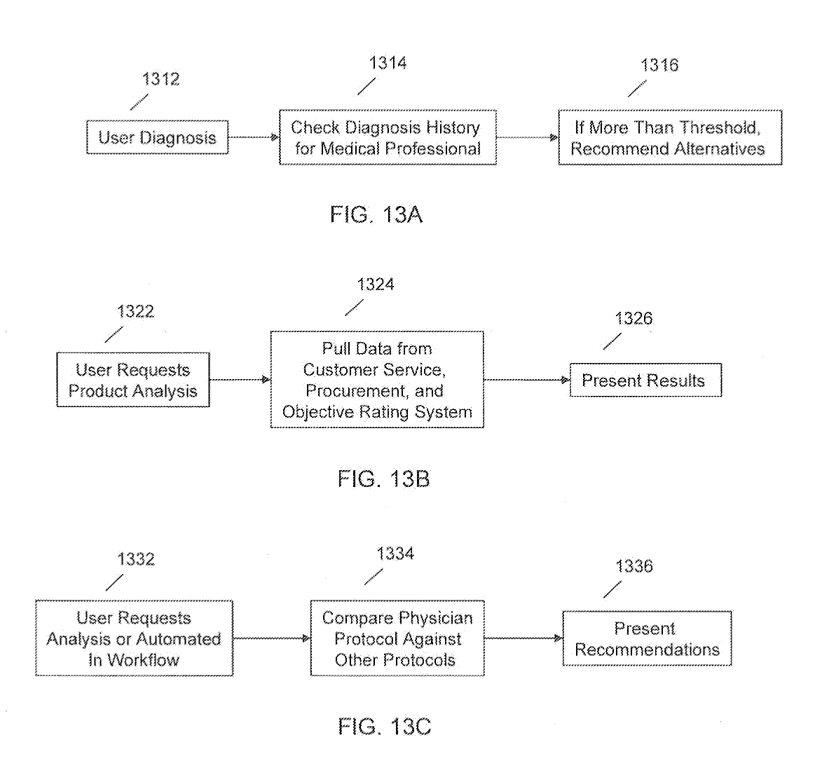

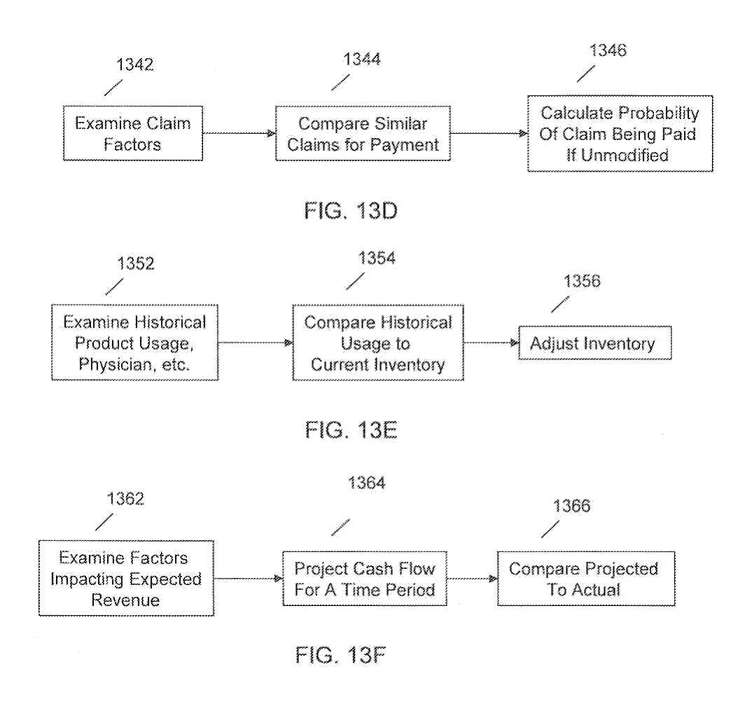

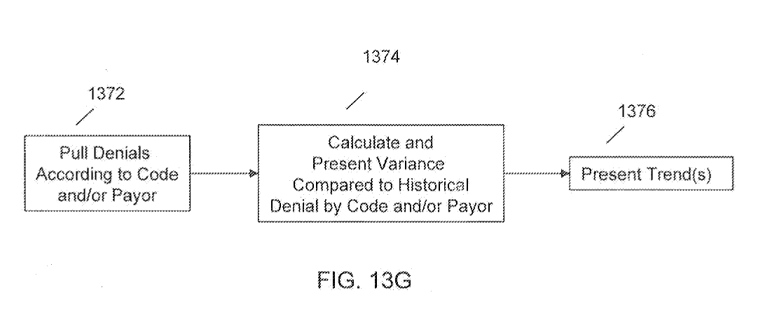

According to another example embodiment as illustrated in FIG. 13(A-G), once a healthcare and workflow management system has been implemented and is operational, other automated service opportunities may be incorporated into the system. Once data has been collected on various aspects of the system through use, enhancement and changes may be made to improve the quality of service and performance of the system.

In one aspect of this example embodiment, illustrated in FIG. 13A, product recommendations based on the user diagnosis may be improved using past diagnosis and recommendations processing. For example, a user diagnosis is supplied 1312 to the system. The system then checks this diagnosis with historical data of the same or similar diagnoses evaluated by a medical professional 1314. If, for example, 10 or more of the same or similar diagnosis have been made within the past 120 days, the system may recommend alternative products that have been selected in those past diagnoses. The date range and number of same or similar diagnosis may be further modified as needed to improve effectiveness. For example, while a range 10 or more same or similar diagnosis is contemplated, the number could ultimately be 5, 10, 25, 50, 100, or greater depending on the application. Similarly, while the contemplated range of previous diagnosis is 120 days, the range could be 30 days, 60 days, 120 days, 6 months, 12 months, or greater depending on the application.

In another embodiment, illustrated in FIG. 13B, the system may perform a product analysis. Here a user may request a product analysis in step 1322 and the system automatically retrieves data from a variety of sources including, but not limited to, customer service, procurement, and from an objective rating system 1324. The data is then manipulated and presented in a meaningful format to the user (for example, matrix, recommendations, et cetera) 1326. For example, sample product factors may include whether OEM product objectives are achieved and can be rated on a scale (for example, poor, fair, good, very good, excellent) based on feedback. Cost to revenue may also be displayed, for example 1:4 may be rated as excellent, 1:3 may be rated as good, and less than 1:2 may be rated as poor. Quality of the product(s) may also be assessed by tracking the number of returns. Here, for example, returns of less than 1% may be considered as excellent, 1-3% may be considered as good, 3-5% may be considered as acceptable, and greater than 5% may be considered as poor. A range of patient usability may also be assessed based on patient feedback and satisfaction scores, and can similarly be ranked on a scale.

In another embodiment, illustrated in FIG. 13C, the system may perform a protocol analysis. Here the user may request a protocol analysis or have it automated in the workflow 1332. The system then compares a first physician's protocol against other physicians' protocols and/or against a standard protocol 1334. The system then presents to the user recommendations by categories such as surgeries, diagnoses, or other meaningful forms 1336.

In another example embodiment, illustrated in FIG. 13D, the system may perform a claim scrub analysis. Here the system examines a present claim in view of various claim factors including, but not limited to, a patient's diagnosis, a patient's risk, a specified payor, et cetera 1342. The system then compares similar claims that have been previously filed for payment 1344. Using historical information from past claims, for example from a proprietary claims data base for example stored as part of data base 214, the system calculates and outputs an indication (for example, a probability) regarding if the present claim is likely to be paid as-is 1346. The system may also suggest changes to the claims that may improve the probability of payment.

In another example embodiment, illustrated in FIG. 13E, the system may perform an inventory predictability analysis. Here the system examines historical product usage by month (or other periods of time), physician activities, trends, and/or other factors 1352. The system then compares the historical usage with a current inventory 1354. After performing the comparison, the present inventory may be increased, decreased, or held the same based on the historical usage 1356.

In another example embodiment, illustrated in FIG. 13F, the system may perform a cash flow analysis. Here the system may examine a variety of factors which may impact expected revenue, including examining average payment turnaround by a payor, examining terms from manufacturers and determining which products need to be replenished or restocked based on a purchase order and PAR levels 1362. An embodiment may then project cash flow for a period of time 1364 by assessing, in addition to expected revenue form a payor such as an insurer, an average time period and amounts where patients are responsible for a payment. An embodiment may also compare actual accounts receivable versus expected 1366, and assess trends in payments.

In another example embodiment, illustrated in FIG. 13F, the system may perform a reimbursement trending analysis. Here the system retrieves denials by a payor and any associated codes or messages, if available, for a given time interval 1372. The system then presents a variance compared to historical denials by the same associated codes or messages 1374. Next, the system may present any significant trends that may be identified, including whether the trend is positive or negative 1376. These different trends can be user defined and adjusted according to the user's needs.

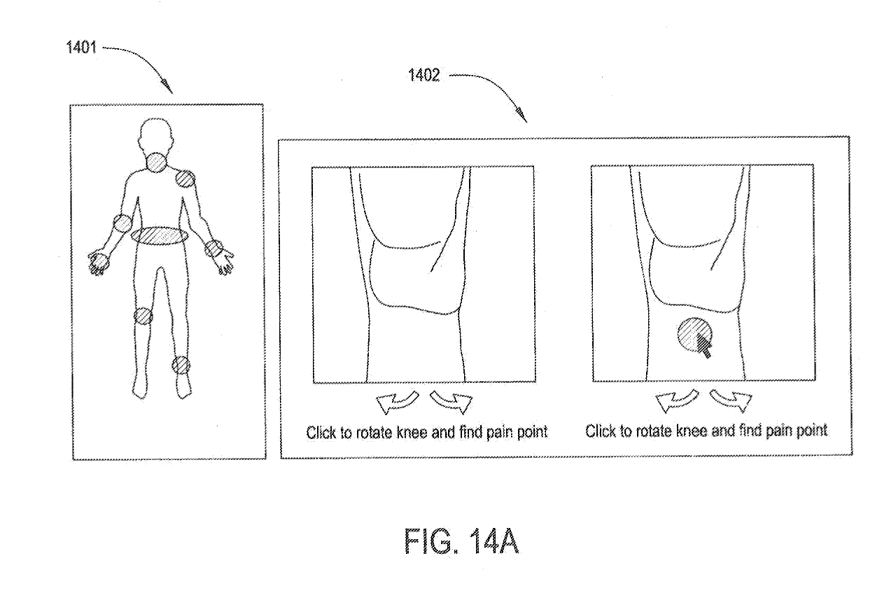

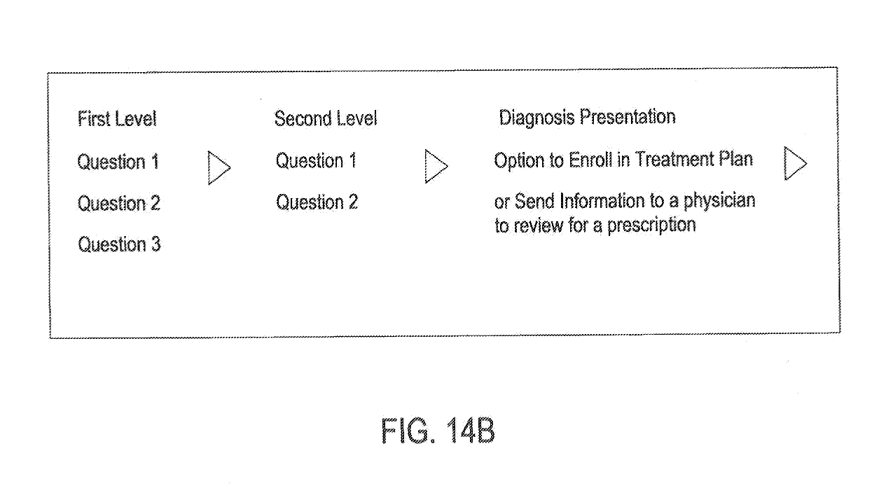

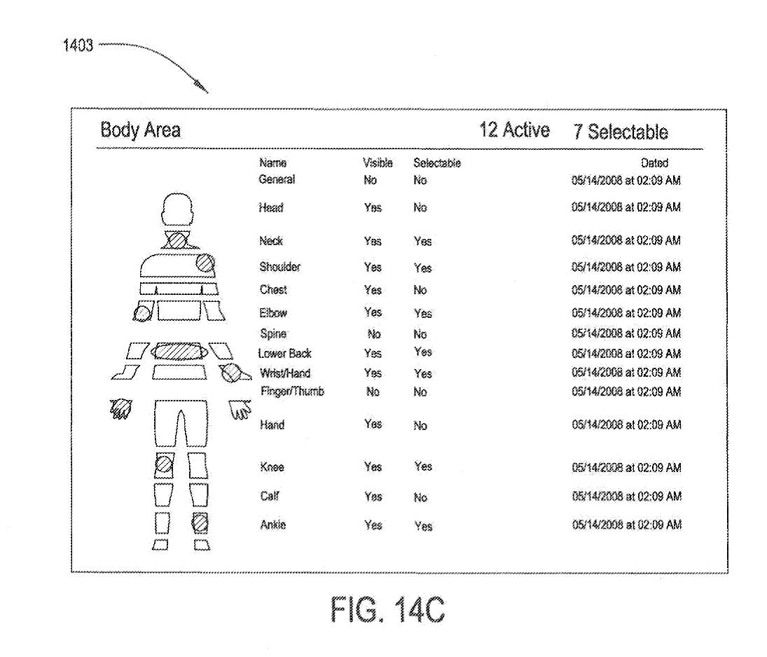

FIG. 14(A-C) illustrates an example of a public interface of system 20, such as facilitated via public interface module 122, and modification mechanisms available for modifying the interface. Public interface may provide, for example in a web browser view or as a downloadable application, an anatomical navigation interface. The anatomical navigation interface may provide gross or high level selections via a top level interface 1401, and may provide more detailed anatomical navigation via a detailed interface 1402. The user may select the gross and detailed areas of concern for an evaluation, such as during a virtual consultation or visit, as described in connection with FIG. 3 or FIG. 4, for example. Prior to, after or in combination with the user operating the anatomical navigation interface, the user may be asked for information, such as in the form of questions and answers. For example, illustrated in FIG. 14B is a question hierarchy, where first level questions may lead to second level questions, which may in turn lead to a diagnosis presentation. Alternatively, the answering of questions, which could include referencing the user to the anatomical navigation interface to enter further detailed information via that interface, may end in sending information to a physician or healthcare provider for review in order to provide a diagnosis and prescription. The public interface, including the anatomical navigation and question tree may be used as part of a virtual visit to enter a diagnosis into the system 20, such that a patient agreement process (formalizing a treatment plan) may be initialized.

In order to drive the functionality of the public interface including anatomical navigation interface, an embodiment employs a question tree (FIG. 14B). The question content and flow (ordering, connectivity with the anatomical navigation tool) may be easily modified by a user such that the program presents the appropriate anatomical navigation and questions for a given condition diagnosis. The appropriate questions and appropriate anatomical navigation may change over time, or may change per physician or healthcare provider.

Accordingly, the question tree may be built dynamically using the anatomical navigation interface and a combination of photos and questions to better understand the user's issue(s) during a virtual visit. The question tree may be refined to the level of an actual diagnosis that can be entered into system 20 for processing, just as if the patient had made an in person visit to a physician or healthcare provider and received a diagnosis. The refinement level can reasonably be provided to the diagnosis level for many musculoskeletal/orthopedic conditions. The system 20 has the capability to require a prescription from a physician prior to further processing of a diagnosis within the system. Alternatively, a treatment plan may be offered in an unscripted form for various conditions. The treatment plan, as described throughout, may be related to musculoskeletal/orthopedic conditions and include exercises, activity restrictions, and may be facilitated by virtual meetings (chat sessions, video chat and the like) with a clinical specialist, physician and/or healthcare provider.

Referring to FIG. 14C, a module provides a tool for dynamically modifying the question tree and/or anatomical navigation interface in a convenient way for a non-programmer As such, the tool provides a screen view 1403 in which a user may select an anatomical area of interest, for example the neck, shoulder, lower back, et cetera. On selection, an editing tool is provided, for example via pop up window, in which a user may modify current question trees and/or anatomical images for the anatomical navigation interface/question tree. The user may modify a question for example via typing new or modified text into at text box for a current question. A user may modify a current ordering of questions by simply dragging or otherwise rearranging the question order (for example via visible slider or changing numerical ordering associated with the questions). Moreover, a user may add a new question or delete an existing question, for example via selecting appropriate check boxes or radio buttons. Similarly, a user may modify, change, reorder, add or delete the anatomical images used in the navigation interface. For example, the user may accomplish this by uploading new or modified photos/drawings in addition to or in lieu of the existing photos/drawings. Also, a user may modify the nature in which the overall flow of the navigational interface/question tree operates by changing the numerical ordering of items in the flow. For example, if the flow is prompt for anatomical selection, followed by question, followed by prompt for anatomical selection, the user may update this to prompt for anatomical selection, prompt for anatomical selection, followed by question by simply ordering the numerical value of the elements. Thus, if the original numerical ordering is (1) prompt for anatomical selection, (2) question, (3) prompt for anatomical selection, a new order may be (1) prompt for anatomical selection, (2) prompt for anatomical selection, (3) question.

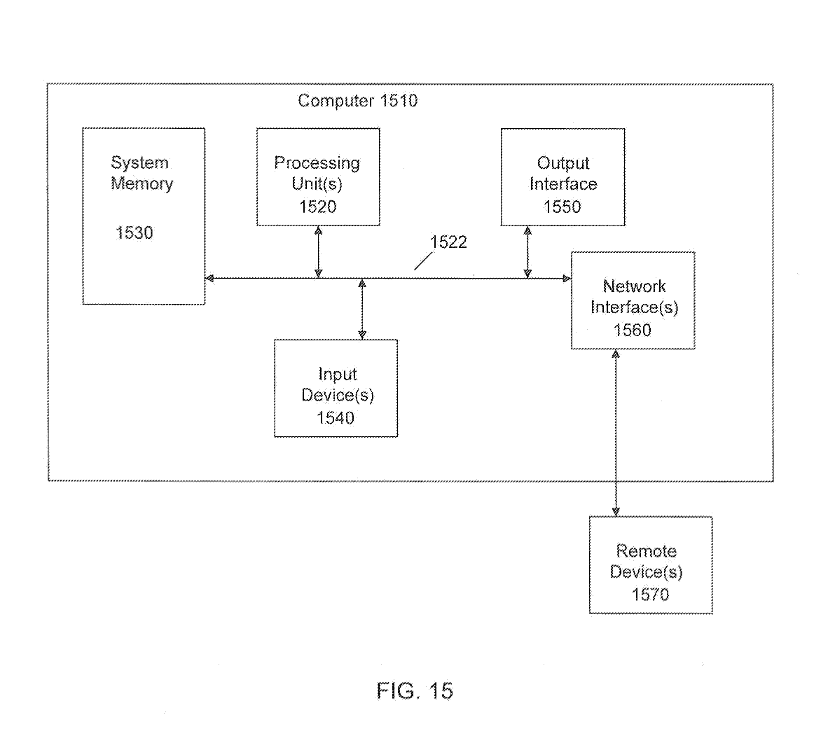

It will be understood by those having ordinary skill in the art that embodiments may be implemented in one or more information handling devices configured appropriately to execute program instructions consistent with the functionality of the embodiments as described herein. In this regard, FIG. 1 and FIG. 15 illustrate non-limiting examples of such devices and components thereof. While servers and mobile computing systems such as tablet computers, laptop computers, and smart phones have been specifically mentioned as examples herein, embodiments may be implemented using other systems or devices, such as desktops, workstations, and the like.

Referring to FIG. 15, it will be readily understood that embodiments may be implemented using any of a wide variety of devices or combinations of devices, for example for implementing functionality as described herein. An example device that may be used in implementing embodiments includes a computing device in the form of a computer 1510. In this regard, the computer 1510 may execute program instructions configured to provide healthcare solutions and workflow management, and perform other functionality of the embodiments, as described herein.

Components of computer 1510 may include, but are not limited to, at least one processing unit 1520, a system memory 1530, and a system bus 1522 that couples various system components including the system memory 1530 to the processing unit(s) 1520. The computer 1510 may include or have access to a variety of computer readable media. The system memory 1530 may include computer readable storage media in the form of volatile and/or nonvolatile memory such as read only memory (ROM) and/or random access memory (RAM). By way of example, and not limitation, system memory 1530 may also include an operating system, application programs, other program modules, and program data.

A user may interface with (for example, enter commands and information) the computer 1510 through input devices 1540. A monitor or other type of device can also be connected to the system bus 1522 via an interface, such as an output interface 1550. In addition to a monitor, computers may also include other peripheral output devices. The computer 1510 may operate in a networked or distributed environment using logical connections (network interface 1560) to other remote computers or databases (remote device(s) 1570), such as for communication between devices comprising system 100. The logical connections may include a network, such local area network (LAN) or a wide area network (WAN), but may also include other networks/buses.

As will be appreciated by one skilled in the art, aspects may be embodied as a system, method or computer program product. Accordingly, aspects of the present invention may take the form of an entirely hardware embodiment, or an embodiment including software (including firmware, resident software, micro-code, et cetera) that may all generally be referred to herein as a “circuit,” “module” or “system.” Furthermore, aspects of the present invention may take the form of a computer program product embodied in at least one computer readable medium(s) having computer readable program code embodied therewith.

Any combination of at least one computer readable medium(s) may be utilized. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible or non-signal medium that can contain or store a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for embodiments may be written in any combination of programming languages, including an object oriented programming language such as Java, Smalltalk, C++ or the like and conventional procedural programming languages, such as the “C” programming language or similar programming languages. The program code may execute entirely on a first computer, partly on the first computer, as a stand-alone software package, partly on the first computer and partly on a remote computer or entirely on the remote computer or server. In the latter scenario, the remote computer may be connected to the first computer through any type of network, including a local area network (LAN) or a wide area network (WAN), or the connection may be made to an external computer (for example, through the Internet using an Internet Service Provider).

Embodiments are described with reference to figures of methods, apparatus (systems) and computer program products according to embodiments. It will be understood that portions of the figures can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Although illustrated example embodiments have been described herein with reference to the accompanying drawings, it is to be understood that embodiments are not limited to those precise example embodiments, and that various other changes and modifications may be affected therein by one skilled in the art without departing from the scope or spirit of the disclosure.

What is claimed is:

1. A computer program product, comprising:
a computer readable storage medium having computer readable program code embodied therewith, the computer readable program code comprising:
computer readable program code configured to, in response to an input physician identifier and an input diagnosis identifier, select a musculoskeletal treatment protocol, the musculoskeletal protocol including at least one of the following: a predetermined musculoskeletal medical product and a predetermined musculoskeletal treatment service;
computer readable program code configured to, responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and
computer readable program code configured to fill at least a portion of the order corresponding to the musculoskeletal treatment protocol.

2. The computer program product of claim 1, further comprising:
computer readable program code configured to verify availability of a predetermined musculoskeletal medical product of said order corresponding to a musculoskeletal treatment protocol by referencing an inventory of musculoskeletal medical products;
wherein to fill at least a portion of the order corresponding to the musculoskeletal treatment protocol includes updating a system inventory status with respect to a predetermined musculoskeletal medical product.

3. The computer program product of claim 1, further comprising:
computer readable program code configured to provide scheduling information regarding a request for a predetermined musculoskeletal treatment of said order corresponding to a musculoskeletal treatment protocol;
wherein to fill at least a portion of the order corresponding to the musculoskeletal treatment protocol includes updating a treatment schedule status with respect to a scheduled treatment for the patient.

4. The computer program product of claim 2, wherein the predetermined musculoskeletal medical product comprises a non-invasive musculoskeletal medical product, wherein the non-invasive musculoskeletal medical product comprises a brace.

5. The computer program product of claim 2, wherein the predetermined musculoskeletal medical product comprises an invasive musculoskeletal medical product, wherein the invasive musculoskeletal medical product comprises an implantable medical product.

6. The computer program product of claim 1, wherein the predetermined musculoskeletal treatment service comprises a non-invasive musculoskeletal treatment regimen of physical therapy.

7. The computer program product of claim 1, wherein the order corresponding to a musculoskeletal treatment protocol includes patient information.

8. The computer program product of claim 7, wherein at least a portion of the patient information is automatically gathered from a medical insurance provider associated with the patient based on an account identification process.

9. The computer program product according to claim 1, further comprising computer readable program code configured to provide a prediction that an insurance provider covers at least one portion of the order.

10. The computer program product according to claim 9, wherein the prediction that an insurance provider covers at least one portion of the order is based on a reference to a proprietary database comprising tacked outcomes of similarly situated patients.

11. The computer program product according to claim 9, further comprising computer readable program code configured to provide an alternate billing recommendation for said order responsive to a prediction that a health insurance provider does not cover at least one portion of the order.

12. The computer program product according to claim 1, further comprising computer readable program code configured to map at least one portion of said order to one or more billing forms of an insurance provider.

13. The computer readable program product according to claim 1, further comprising computer readable program code configured to track an outcome with respect to said patient responsive to implementing said musculoskeletal treatment protocol.

14. The computer program product according to claim 13, further comprising computer readable program code configured to provide one or more alternatives to said musculoskeletal treatment protocol responsive taking into account outcomes of one or more patients previously undergoing a similar musculoskeletal treatment protocol.

15. The computer program product according to claim 1, further comprising:
computer readable program code configured to receive patient input information.

16. The computer program product of claim 15, wherein the patient input information comprises patient input information provided over a network connection.

17. The computer program product of claim 16, wherein the patient input information comprises one of image data, video data, and anatomical navigation input data.

18. The computer program product of claim 17, further comprising:
computer readable program code configured to permit near real-time patient and healthcare provider interaction; and
computer readable program code configured to permit submission of a prescription to a third party responsive to said near real-time patient and healthcare provider interaction.

19. A method, comprising:
operating a computing device to, in response to an input physician identifier and an input diagnosis identifier, select a musculoskeletal treatment protocol, the musculoskeletal treatment protocol including at least one of the following: a predetermined musculoskeletal medical product and a predetermined musculoskeletal treatment service;
operating the computing device to, responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and
operating the computing device to fill at least a portion of the order corresponding to the musculoskeletal treatment protocol.

20. An apparatus, comprising:
one or more processors;
a storage medium in connection with the one or more processors, the storage medium storing a program of instructions that when executed by the one or more processors causes the apparatus to:
in response to an input physician identifier and an input diagnosis identifier, select a musculoskeletal treatment protocol, the musculoskeletal treatment protocol including at least one of the following: a predetermined musculoskeletal medical product and a predetermined musculoskeletal treatment service;
responsive to selecting a musculoskeletal treatment protocol, produce an order corresponding to the musculoskeletal treatment protocol on behalf of a patient, wherein the order corresponding to a musculoskeletal treatment protocol includes at least one of a request for a predetermined musculoskeletal medical product and a request for a predetermined musculoskeletal treatment service; and
fill at least a portion of the order corresponding to the musculoskeletal treatment protocol.

21. Systems, products, apparatuses and methods as shown and described.
